(19) United States
(12) Patent Application Publication
(10) Pub. No.: US 2021/0024873 A1
(43) Pub. Date: Jan. 28, 2021

(54) REAL-TIME MONITORING OF PROTEIN CONCENTRATION USING ULTRAVIOLET SIGNAL

(71) Applicant: BRISTOL-MYERS SQUIBB COMPANY, Princeton, NJ (US)

(72) Inventors: Deqiang YU, Sharon, MA (US); Xiaoming LIU, Acton, MA (US); Chao MA, Leominster, MA (US); Jongchan LEE, Carlisle, MA (US); Zhengjian LI, Sudbury, MA (US)

(21) Appl. No.: 17/041,851

(22) PCT Filed: Mar. 26, 2019
(86) PCT No.: PCT/US2019/024132

§ 371 Date: Sep. 25, 2020

Related U.S. Application Data
(60) Provisional application No. 62/648,752, filed on Mar. 27, 2018.
(60) Provisional application No. 62/667,250, filed on May 4, 2018.

(51) Int. Cl. C12M 1/34; C12M 1/00; C07K 1/34; C07K 16/00; G01N 21/79; G01N 33/52

(52) U.S. Cl. CPC C12M 41/32; C12M 47/10; G01N 33/52; C07K 16/00; G01N 21/79; C07K 1/34

(57) ABSTRACT

Disclosed herein are methods of controlling, modulating, increasing, or improving protein yield in a sample mixture comprising a target protein and impurities comprising monitoring in real-time an ultraviolet (UV) signal of the sample mixture during protein filtration in a harvest skid.

### FIELD OF THE DISCLOSURE

The present disclosure is related to a method of monitoring concentration of a biological molecule, e.g., a protein, in a composition. Specifically, the present disclosure is directed to a method of monitoring, controlling, modulating, or increasing protein yield from a composition using real-time ultraviolet signal during protein filtration.

### BACKGROUND

Hundreds of therapeutic proteins (e.g., monoclonal antibodies (mAbs)) are currently in development, and many companies have multiple antibodies in their pipelines. Basic unit operations such as harvest, Protein A affinity chromatography and additional polishing steps are utilized to purify the protein of interest.

The upstream and recovery operations aims for high productivity of therapeutic protein in both cell culture and recovery process and various on-line configurations are available to monitor bioprocessing operations. See, Whitford W., Julien C. Bioprocess Int. (5), S32-S45 (2007). Recently real-time monitoring and controlling of cell culture process has been implemented. It has been shown that an increase in the non-viable sub-population in CHO cell culture can predict the onset of stationary phase, demonstrating the opportunity for a completely automated cell culture process as well as a reliable and reproducible control of fed-batch additions during culture expansion. Sitton G., Srienc F. J. Biotechnol., 135 (2008), 174-180. Others have utilized multiple steps in the primary recovery process to remove biomass and clarify the feed stream for downstream column chromatography. Bink L. R., Furey J. BioProcess Int. 8(3) 2010, 44-49, 57 (2010).

Some have approached the issue of improving protein yield by addressing upstream steps to increase downstream yield. For example, others attempted to lower mechanical stress to CHO cells caused by the magnetically levitated bearingless centrifugal pumps by using peristaltic and diaphragm pumps. Blaschczok K., et al. Chemie Ingenieur Technik, (85), 144-152 (2013). Still, others have evaluated the proteomic approach by investigating the dynamics and fate of host cell proteins in the supernatant of a monoclonal antibodies producing cell line during recovery and early downstream processing including centrifugation, depth filtration, and Protein A capture chromatography. Hogwood, C. E. M., et al. Biotechnol. Bioeng. 2013(110), 240-251. However, some processes require additional steps, such as fluorescent labeling, to identify protein concentration and yield during the purification process. Ignatova and Gierasch, Proc Natl Acad Sci U S A.; 101(2):523-8 (2004). Adding additional impurities might necessitate additional purification steps that could affect yield.

Thus, there remains a need of real-time monitoring and controlling of recovery process to increase recovery yield and process robustness, quickly evaluate upstream performance and facilitate immediate downstream processing in either batch process or more critically in continuous process.

### SUMMARY

Disclosed herein are novel real-time monitoring and controlling process and system, designed and examined for a filtration based cell culture harvesting process, e.g., depth filtration harvest, of several therapeutic proteins. Methods described herein provide several advantages over the art. First, the design of harvest skid has capabilities of real-time monitoring and controlling of critical process parameters and quality attributes. Second, it translates online UV signal of clarified bulk to real-time titer of target product using modeling methods. Third, use of this harvest skid and real-time titer automatically controls harvest process and improve process yield, robustness and consistency. Finally, use the titer information to demonstrate cell culture performance and guide the immediate processing of downstream purification.

Core of this new technology is the application of real-time monitoring of UV signal during harvest process, and translation of online UV signal to real-time target protein concentration. The model disclosed herein can be applied to several processes with different cell properties and productivity level. With this system, start and end of clarified bulk collection can be determined in a quantitative way, which significantly improves harvest robustness and protein yield.

The methods disclosed herein provide a deep insight into the application of a harvest skid in cell culture clarification process. The new harvest process disclosed herein improve protein yield while being scalable, auto-controllable, and applicable for multi-product with a wide range of properties. The real-time titer information can be used to demonstrate cell culture performance and guide immediate downstream processing.

Disclosed herein is a method of monitoring in real-time a target protein concentration (titer) in a sample mixture comprising a target protein and impurities comprising monitoring in real-time an ultraviolet (UV) signal of the sample mixture and automatically transferring the UV signal into target protein titer using established models during a filtration based cell culture harvesting process.

Also disclosed herein is a method of controlling target protein collection and improving protein yield in a sample mixture comprising a target protein and impurities comprising monitoring in real-time an ultraviolet (UV) signal of the sample mixture during a filtration based cell culture harvesting process.

In some embodiments, the UV signal is continuously transferred to a titer of the target protein according to established models and automatic control.

In some embodiments, the titer of the target protein is at least about 0.01 g/L, at least about 0.02 g/L, at least about 0.03 g/L, at least about 0.04 g/L, at least about 0.05 g/L, at least about 0.06 g/L, at least about 0.07 g/L, at least about 0.08 g/L, at least about 0.09 g/L, at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 6.5 g/L, at least about 7 g/L, at least about 7.5 g/L, at least about 8 g/L, at least about 8.5 g/L, at least about 9 g/L, at least about 9.5 g/L, at least about 10 g/L, at least about 10.5 g/L, at least about 11 g/L, at least about 11.5 g/L, at least about 12 g/L, at least about 12.5 g/L, at least about 13 g/L, at least about 13.5 g/L, at least about 14 g/L, at least about 14.5 g/L, at least about 15 g/L, at least about 15.5 g/L, at least about 16 g/L, at least about 16.5 g/L, at least about 17 g/L, at least about 17.5 g/L, at least about 18 g/L, at least about 18.5 g/L, at least about 19 g/L, at least about 19.5 g/L, or at least about 20 g/L.

In some embodiments, the methods disclosed herein further comprise beginning collection of the target protein when the titer is at least about 0.05 g/L, at least about 0.06 g/L, at least about 0.07 g/L, at least about 0.08 g/L, at least about 0.09 g/L, at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 6.5 g/L, at least about 7 g/L, at least about 7.5 g/L, at least about 8 g/L, at least about 8.5 g/L, at least about 9 g/L, at least about 9.5 g/L, at least about 10 g/L, at least about 10.5 g/L, at least about 11 g/L, at least about 11.5 g/L, at least about 12 g/L, at least about 12.5 g/L, at least about 13 g/L, at least about 13.5 g/L, at least about 14 g/L, at least about 14.5 g/L, at least about 15 g/L, at least about 15.5 g/L, at least about 16 g/L, at least about 16.5 g/L, at least about 17 g/L, at least about 17.5 g/L, at least about 18 g/L, at least about 18.5 g/L, at least about 19 g/L, at least about 19.5 g/L, or at least about 20 g/L.

In some embodiments, the titer that the target protein is collected is between about 0.05 g/L and about 20 g/L, between about 0.1 g/L and about 20 g/L, between about 0.2 g/L and about 20 g/L, between about 0.3 g/L and about 20 g/L, between about 0.4 g/L and about 20 g/L, between about 0.5 g/L and about 20 g/L, between about 0.6 g/L and about 20 g/L, between about 0.7 g/L and about 20 g/L, between about 0.8 g/L and about 20 g/L, between about 0.9 g/L and about 20 g/L, between about 1 g/L and about 20 g/L, between about 0.05 g/L and about 15 g/L, between about 0.1 g/L and about 15 g/L, between about 0.2 g/L and about 15 g/L, between about 0.3 g/L and about 15 g/L, between about 0.4 g/L and about 15 g/L, between about 0.5 g/L and about 15 g/L, between about 0.6 g/L and about 15 g/L, between about 0.7 g/L and about 15 g/L, between about 0.8 g/L and about 15 g/L, between about 0.9 g/L and about 15 g/L, or between about 1 g/L and about 15 g/L, between about 0.05 g/L and about 10 g/L, between about 0.1 g/L and about 10 g/L, between about 0.2 g/L and about 10 g/L, between about 0.3 g/L and about 10 g/L, between about 0.4 g/L and about 10 g/L, between about 0.5 g/L and about 10 g/L, between about 0.6 g/L and about 10 g/L, between about 0.7 g/L and about 10 g/L, between about 0.8 g/L and about 10 g/L, between about 0.9 g/L and about 10 g/L, or between about 1 g/L and about 10 g/L.

In some embodiments, the methods disclosed herein further comprise stopping the collection of the target protein when the collection titer is below about 0.1 or 0.2 g/L.

In some embodiments, the target protein yield is increased at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% compared to the protein yield without monitoring in real time an ultraviolet (UV) signal of the sample mixture.

In some embodiments, the target protein is harvested from a culture medium having a cell density of at least about 1×10⁶ cells/mL, at least about 5×10⁶ cells/mL, at least about 1×10⁷ cells/mL, at least about 1.5×10⁷ cells/mL, at least about 2×10⁷ cells/mL, at least about 2.5×10⁷ cells/mL, at least about 3×10⁷ cells/mL, at least about 3.5×10⁷ cells/mL, at least about 4×10⁷ cells/mL, at least about 4.5×10⁷ cells/mL, or at least about 5×10⁷ cells/mL.

In some embodiments, the protein filtration is a depth filtration. In some embodiments, the depth filtration comprises a primary depth filter and/or a secondary depth filter.

In some embodiments, the methods disclosed herein further comprise loading the sample mixture prior to the monitoring. In some embodiments, the methods disclosed herein further comprise flushing the depth filters with water or buffer before loading the cell culture and chasing the depth filters post loading the cell culture. In some embodiments, the methods disclosed herein further comprise chasing the sample mixture with phosphate buffered saline (PBS) or other buffers. In some embodiments, the filtration based cell culture harvesting process comprises a harvest skid. In some embodiments, the harvest skid comprises a control system wherein the control system automatically starts collection of the protein when the set titer is achieved. In some embodiments, the harvest skid comprises a control system, wherein the control system automatically stops collection of the protein when the set titer is achieved. In some embodiments, the control system modulates flow rate of a liquid through the harvest skid. In some embodiments, the control system automatically drives the pump to up-regulate flow rate through the harvest skid. In some embodiments, the control system automatically drives the pump to down-regulate flow rate through the harvest skid. In some embodiments, the methods disclosed herein do not comprise a step of air blow-down. In some embodiments, the target protein titer or the protein yield is not based on volume.

In some embodiments, disclosed herein is a method of increasing, controlling, or modulating protein yield in a sample mixture comprising a target protein and impurities comprising (a) flushing a harvest skid with water; (b) loading the sample into the harvest skid; (c) measuring an ultraviolet (UV) signal of the sample mixture during protein filtration in the harvest skid into a real-time protein titer; (d) starting collection of the protein based on the UV measurement and the real-time protein titer; (e) chasing the protein with PBS; and (f) stopping collection of the protein based on the UV measurement and the real-time protein titer; wherein the UV signal correlates with the real-time protein titer during the filtration.

In some embodiments, the methods further comprise measuring pressure, turbidity, temperature, flow rate, or any combination thereof.

In some embodiments, the methods further comprise measuring pressure using a pressure sensor. In some embodiments, the pressure is measured in a range of −10 pounds per square inch (psi) to 50 psi, −10 psi to 40 psi, −9 psi to 40 psi, −8 psi to 40 psi, −7 psi to 30 psi, −6 psi to −20 psi, −7 psi to 40 psi, −8 psi to 40 psi, −9 psi to 45 psi, −10 psi to −45 psi, or −7 psi to −45 psi.

In some embodiments, the methods further comprise measuring turbidity. In some embodiments, the turbidity is measured in a range of 0 absorbance units (AU) to 2 AU.

In some embodiments, the methods further comprise measuring temperature. In some embodiments, the temperature is measured in a range of 0° C. to 70° C., 0° C. to 60° C., 0° C. to 50° C., 0° C. to 40° C., 5° C. to 70° C., 10° C. to 70° C., 15° C. to 70° C., 20° C. to 70° C., 10° C. to 60° C., 20° C. to 50° C., 20° C. to 40° C., 20° C. to 45° C., 30° C. to 40° C., 35° C. to 40° C., 20° C. to 30° C., 35° C. to 40° C., or 25° C. to 45° C,.

In some embodiments, the methods further comprise measuring flow. In some embodiments, the flow is measured in a range of 0 L/min to 20 L/min, .0 L/min to 30 L/min, 0 L/min to 40 L/min, 0 L/min to 50 L/min, 0 L/min to 60 L/min, 0 L/min to 70 L/min, 0 L/min to 80 L/min, 0 L/min to 90 L/min, 0 L/min to 100 L/min, 0 L/min to 110 L/min, 0 L/min to 120 L/min, 0 L/min to 130 L/min, 0 L/min to 140 L/min, 0 L/min to 150 L/min, 0 L/min to 160 L/min, 0 L/min to 170 L/min, 0 L/min to 180 L/min, 0 L/min to 190 L/min, 0 L/min to 200 L/min, 0 L/min to 250 L/min, or 0 L/min to 300 L/min.

In some embodiments, the harvest skid comprises one or more filters. In some embodiments, the filters comprise a primary depth filter and a secondary depth filter. In some embodiments, the sample mixture is selected from the group consisting of a pure protein sample, a clarified bulk protein sample, a cell culture sample, and any combination thereof.

In some embodiments, the protein is produced in culture comprising mammalian cells. In some embodiments, the mammalian cells are Chinese hamster ovary (CHO) cells, HEK293 cells, mouse myeloma (NS0), baby hamster kidney cells (BHK), monkey kidney fibroblast cells (COS-7), Madin-Darby bovine kidney cells (MDBK) or any combination thereof.

In some embodiments, the protein comprises an antibody or a fusion protein. In some embodiments, the protein is an anti-GITR antibody, an anti-CXCR4 antibody, an anti-CD73 antibody, an anti-TIGIT antibody, an anti-OX40 antibody, an anti-LAG3 antibody and anti-IL8 antibody. In some embodiments, the protein is Abatacept or Belatacept.

In some embodiments, disclosed herein is a system for real time monitoring and controlling of protein yield, wherein the system comprises a sensor measuring a real-time UV signal of a sample mixture comprising a target protein and impurities.

In some embodiments, the system further comprises a sensor measuring pressure, turbidity, temperature, flow, weight, or any combination thereof.

In some embodiments, an apparatus comprises a sensor configured to measure a UV signal of a sample mixture comprising a target protein and impurities. In some embodiments, the processor is configured to control collection of the target protein. In some embodiments, the processor is configured to use a target protein titer. In some embodiments, the processor is configured to use established models to determine a cell culture harvest process. In some embodiments, the cell culture harvesting process comprises a filtration based cell culture harvesting process. In some embodiments, a system comprises an apparatus comprising a sensor configured to measure a UV signal of a sample mixture comprising a target protein and impurities.

In some embodiments, the system disclosed is for use in the methods described herein.

### BRIEF DESCRIPTION OF THE DRAWINGS

FIG. 1A shows mechanical design of an exemplary harvest skid. All values are listed in inches. FIG. 1B shows the physical picture of harvest skid.

FIG. 2 shows process flow chart for cell culture harvest process with new harvest skid. Various boxes illustrate online measurement sensors, control modules and physical instruments.

FIG. 3 shows the experimental design described herein to model UV signal to product titer.

FIG. 4 shows a graphic comparison between old and new harvest methods. Compared with former methods, the new method eliminates air blow-down step. Meanwhile, start and end of collections of clarified bulk in the new method can be controlled automatically based on online UV readings and calculated titer. More specifically, real-time target protein concentration during the harvest process can be calculated by online UV sensor readings, using the models generated and tested herein. Cut-off of bulk collection can therefore be determined directly on calculated online target protein concentration. The calculation algorithms can be integrated into Delta V control system to achieve automatic cut-off of clarified bulk collection.

FIG. 5 shows offline titer measurements against online UV signals using serial dilution samples of GITR cell culture.

FIG. 6A and FIG. 6B show offline titer measurements against online UV signals with small scale harvest processes using pure protein (FIG. 6A) and clarified bulk (FIG. 6B). Online UV and offline titer values during the test harvest process were plotted.

FIG. 7A, FIG. 7B, and FIG. 7C show offline titer measurements against online

UV signals with large scale harvest processes using an anti-GITR antibody cell culture (FIG. 7A), Abatacept cell culture (FIG. 7B), and anti-CXCR4 antibody cell culture (FIG. 7C).

FIG. 8A and FIG. 8B show linear fit of offline titer measurements against online

UV values (FIG. 8A); Linear fit of predicted titer based on UV against actual titer (FIG. 8B).

FIG. 9A and FIG. 9B show non-linear fit of offline titer measurements against online UV values (FIG. 9A); Linear fit of predicted titer based on UV against actual titer (FIG. 9B).

FIG. 10 shows mean difference between model-predicted titer and actual titer (HPLC analyzed) for seven molecules studied, including Aba J, anti-CD73 antibody, anti-GITR antibody, anti-IL8 antibody, anti-CXCR4 antibody, anti-OX40 antibody, and anti-TIGIT antibody.

FIG. 11 shows the comparison of online UV trace, titer trace achieved by modelling from UV signal, and titer determined offline. The Y axis shows titer (g/L) determined offline or titer modelled based on UV (g/L), and the X axis shows time (min). The triangle line shows online UV, the square line shows titer modelled based on UV (g/L), and the diamond line shows offline titer (g/L).

### DETAILED DESCRIPTION

Various methods are provided which can be employed to control, modulate, or increase protein yield. Methods include controlling, modulating, or increasing protein yield using real-time measurement of an ultraviolet (UV) signal of a sample mixture during a purification step, e.g., protein filtration in a harvest skid. The methods utilize a UV signal to provide a titer of the target protein according to formulae disclosed herein, which differ based on whether collection occurs from the start of loading to the end of loading or after the end of loading.

Also disclosed herein are various systems and devices relating to the methods provided herein.
a. Terminology

It is to be noted that the term “a” or “an” entity refers to one or more of that entity;

for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

“Modeling,” or “protein modeling” refers to the method of establishing a linear fit to determine the titer (e.g., in g/L) of a test protein. In one embodiment, modeling includes methods from start collection to end loading (e.g., incline modeling). In another embodiment, modeling includes start chasing to end collection (e.g., decline modeling). In other embodiments, modeling includes both the incline modeling and decline modeling.

“Protein yield,” or “yield” refers to the total amount of protein recovered after the processes disclosed herein. Protein yield can be measured in grams or in a final concentration (e.g., mg/ml) in a fixed volume. Percent yield can also be measures as a percentage of the amount of the starting protein (e.g., bulk enzyme).

The term “controlling protein yield” as used herein can refer to regulating, testing, or verifying the end product (e.g., a protein) collected during the processes disclosed herein. In some embodiments, controlling protein yield is achieved through altering the UV signal in real-time to affect critical process parameters and quality attributes and regulate protein yield. In some embodiments, controlling protein yield refers to maintaining a constant UV signal during the methods disclosed herein in order to achieve a desired protein yield.

The term “modulating protein yield” as used herein refers to changing, varying, or altering the end product (e.g., a protein) collected during the processes disclosed herein. Modulating the protein yield alters the protein end-product yield, which can be increased, reduced, or inhibited. In some embodiments, the process modulates the protein yield, which results in an increase in protein yield. In some embodiments, modulating protein yield is achieved through altering the UV signal in real-time to affect critical process parameters and quality attributes and regulate protein yield.

A harvest skid as described herein comprising multiple sensors for real-time clarification and protein yield increase. A harvest skid, or “skid,” comprises one or more pressure sensors, one or more flow sensors, one or more ultraviolet (UV) sensors, one or more weight sensors, one or more turbidity sensors, and/or one or more temperature sensors,

“Titer” refers to the amount, or the concentration, of a substance in a solution. Titer is determined using both incline modeling and decline modeling as described herein.

As used herein, the terms “ug” and “uM” are used interchangeably with “μg” and “μM,” respectively.

Various aspects described herein are described in further detail in the following subsections.
b. Methods and Uses

The present disclosure is based on the capabilities of real-time UV monitoring and controlling of critical process parameters and quality attributes. The present methods allow translation of online UV signal of clarified bulk to real-time titer of target product using modeling methods. The present methods can then be used to automatically control harvest process and to improve process yield, robustness, and consistency. The titer information can also be used to demonstrate cell culture performance and guide the immediate processing of downstream purification. In some embodiments, disclosed herein is a method of controlling or modulating protein yield in a sample mixture comprising a target protein and impurities comprising monitoring in real-time an ultraviolet (UV) signal of the sample mixture during protein filtration in a harvest skid.

In one embodiment, the disclosure includes a method of monitoring in real-time a target protein concentration (titer) in a sample mixture comprising a target protein and impurities comprising monitoring in real-time an ultraviolet (UV) signal of the sample mixture and automatically transferring the UV signal into target protein titer using established models during a filtration based cell culture harvesting process. In another embodiment, the disclosure provides a method of controlling target protein collection and improving protein yield in a sample mixture comprising a target protein and impurities comprising monitoring in real-time an ultraviolet (UV) signal of the sample mixture during a filtration based cell culture harvesting process.

Also disclosed herein is a method of increasing or improving protein yield in a sample mixture comprising a target protein and impurities comprising monitoring in real-time an ultraviolet (UV) signal of the sample mixture during a filtration based cell culture harvesting process, e.g., protein filtration in a harvest skid.

Protein harvest/purification includes multiple steps to isolate or purify a target protein from the mixture of the protein with impurities, such as cells, cell culture medium, DNA, RNA, other proteins, etc. Clarifying cell culture broth can be the first downstream unit operation in an elaborate sequence of steps required to purify a target protein. A combination of centrifugation and/or filtration, e.g., depth filtration, is used for that operation. The availability of large scale, filtration technology, e.g., depth filtration, that can monitor real-time protein concentration can thus provide the capability to improve and simplify downstream processes.

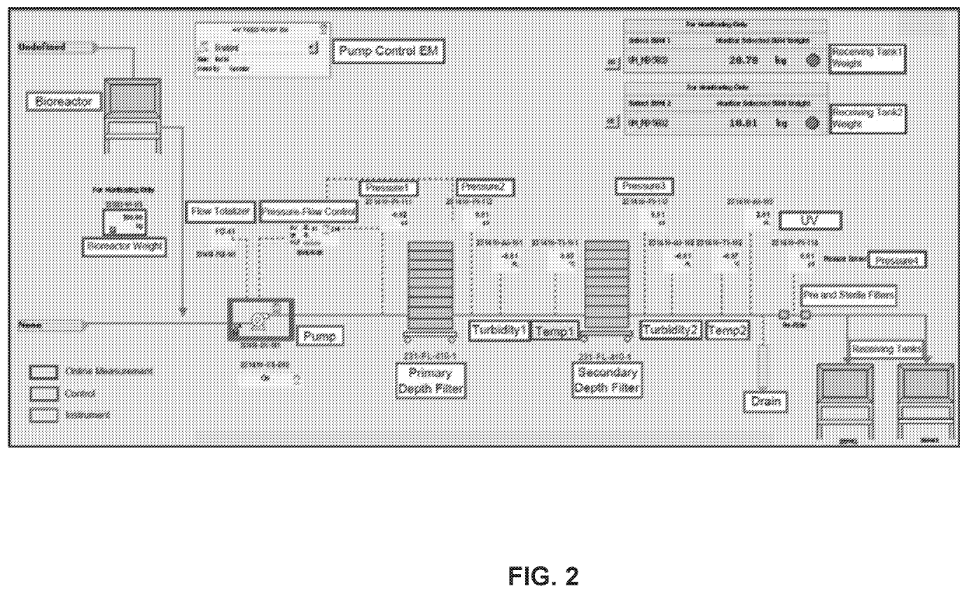

Large-scale depth filtration systems are common in the bioprocess industry. In some embodiments, the depth filtration system can utilize a harvest skid as shown in FIG. 2. Before harvest, depth filters are flushed with water or an appropriate buffer to remove loose particulates and extractables from the filter manufacturing process. The harvest skid can comprise one filter or multiple filters, e.g., a primary depth filter and a second depth filter. Cell culture medium including a target protein can be obtained from a bioreactor and can be loaded onto (or pumped onto) one or more filters, e.g., a primary filter and a secondary filter. The real-time UV signal can be then measured after the loaded cell culture medium has passed through the filter system, e.g., primary filter or the secondary filter. The filtered product can then be obtained at one or more tank. Once a harvest is completed, the filters are again flushed to recover valuable product held-up in the housings. Harvest yields between 50% and 90% can be achievable using a post-use flush and ensuring minimum product loss. The present methods are thus intended to improve the protein harvest yields at least by 1%, at least by 2%, at least by 3%, at least by 4%, at least by 5%, at least by 6%, at least by 7%, at least by 8%, at least by 9%, at least by 10%, at least by 11%, at least by 12%, at least by 13%, at least by 14%, at least by 15%, at least by 16%, at least by 17%, at least by 18%, at least by 19%, at least by 20%, at least by 21%, at least by 22%, at least by 23%, at least by 24%, or at least by 25%.

In some embodiments, the UV signal provides a titer of the target protein from the start of the loading to the end of the loading and/or after the end of the loading till the end of filtration. In some embodiments, the titer of the target proteins from the start of the loading to the end of the loading can be calculated according to formula (I):

Model Predicted Titer=a+b*(online UV signal). (I)

In some embodiments, the titer of the target proteins from the start of the loading to the end of the loading can be calculated according to formula (I), which comprises constants (a) and (b).

In some embodiments, (a) is a value between 0 and −1.0. In some embodiments, (a) is a value between −0.1 and −0.9. In some embodiments, (a) is a value between −0.2 and −0.8. In some embodiments, (a) is a value between −0.3 and −0.7. In some embodiments, (a) is a value between −0.4 and −0.6.

In some embodiments, (a) is a value between −0.2 and −0.5. In some embodiments, (a) is a value between −0.25 and −0.45. In some embodiments, (a) is a value between −0.30 and −0.40.

In some embodiments, (a) is a value between −0.5 and −0.9. In some embodiments, (a) is a value between −0.55 and −0.85. In some embodiments, (a) is a value between −0.60 and −0.80. In some embodiments, (a) is a value between −0.65 and −0.75.

In some embodiments, (a) is about −0.1. In some embodiments, (a) is about −0.15.

In some embodiments, (a) is about −0.2. In some embodiments, (a) is about −0.25. In some embodiments, (a) is about −0.3. In some embodiments, (a) is about −0.35. In some embodiments, (a) is about −0.4. In some embodiments, (a) is about −0.45. In some embodiments, (a) is about −0.5. In some embodiments, (a) is about −0.55. In some embodiments, (a) is about −0.6. In some embodiments, (a) is about −0.65. In some embodiments, (a) is about −0.7. In some embodiments, (a) is about −0.75. In some embodiments, (a) is about −0.8. In some embodiments, (a) is about −0.85. In some embodiments, (a) is about −0.9. In some embodiments, (a) is about −0.95. In some embodiments, (a) is about −1.0.

In some embodiments, (a) is −0.35. In some embodiments, (a) is −0.69. In one embodiment, the cell type is DG44 and (a) is −0.35. In one embodiment, the cell type is CHOZN, and (a) is −0.69.

In some embodiments, (b) is a value between 1.0 and 5.0. In some embodiments, (b) is a value between 1.5 and 4.5. In some embodiments, (b) is a value between 2.0 and 4.0. In some embodiments, (b) is a value between 2.5 and 3.5.

In some embodiments, (b) is a value between 2.0 and 3.6. In some embodiments, (b) is a value between 2.1 and 3.5. In some embodiments, (b) is a value between 2.2 and 3.4. In some embodiments, (b) is a value between 2.3 and 3.3. In some embodiments, (b) is a value between 2.4 and 3.2. In some embodiments, (b) is a value between 2.5 and 3.1. In some embodiments, (b) is a value between 2.6 and 3.0. In some embodiments, (b) is a value between 2.7 and 2.9.

In some embodiments, (b) is a value between 3.3 and 4.8. In some embodiments, (b) is a value between 3.4 and 4.7. In some embodiments, (b) is a value between 3.5 and 4.6. In some embodiments, (b) is a value between 3.6 and 4.5. In some embodiments, (b) is a value between 3.7 and 4.4. In some embodiments, (b) is a value between 3.8 and 4.3. In some embodiments, (b) is a value between 3.9 and 4.2. In some embodiments, (b) is a value between 4.0 and 4.1.

In some embodiments, (b) is about 2.0. In some embodiments, (b) is about 2.1. In some embodiments, (b) is about 2.2. In some embodiments, (b) is about 2.3. In some embodiments, (b) is about 2.4. In some embodiments, (b) is about 2.5. In some embodiments, (b) is about 2.6. In some embodiments, (b) is about 2.7. In some embodiments, (b) is about 2.8. In some embodiments, (b) is about 2.9. In some embodiments, (b) is about 3.0. In some embodiments, (b) is about 3.1. In some embodiments, (b) is about 3.2. In some embodiments, (b) is about 3.3. In some embodiments, (b) is about 3.4. In some embodiments, (b) is about 3.5. In some embodiments, (b) is about 3.6. In some embodiments, (b) is about 3.7. In some embodiments, (b) is about 3.8. In some embodiments, (b) is about 3.9. In some embodiments, (b) is about 4.0. In some embodiments, (b) is about 4.1. In some embodiments, (b) is about 4.2. In some embodiments, (b) is about 4.3. In some embodiments, (b) is about 4.4. In some embodiments, (b) is about 4.5. In some embodiments, (b) is about 4.6. In some embodiments, (b) is about 4.7. In some embodiments, (b) is about 4.8. In some embodiments, (b) is about 4.9. In some embodiments, (b) is about 5.0.

In some embodiments, (b) is 2.88. In some embodiments, (b) is 4.06. In one embodiment, the cell type is DG44 and (b) is 2.88. In one embodiment, the cell type is CHOZN, and (b) is 4.06. In some embodiments, (a) is −0.35 and (b) is 2.88. In some embodiments, (a) is −0.69 and (b) is 4.06. In one embodiment, the cell type is DG44, and (a) is −0.35 and (b) is 2.88. In one embodiment, the cell type is CHOZN, and (a) is −0.69 and (b) is 4.06.

In other embodiments, the titer of the target proteins after the end of the loading till the end of filtration can be calculated according to formula (II):

Model Predicted Titer=A+B*exp(C*online UV signal). (II)

In some embodiments, the titer of the target proteins from the start of the loading to the end of the loading can be calculated according to formula (II), which comprises constants (A), (B), and (C).

In some embodiments, (A) is a value between −2.5 and 1.0. In some embodiments,

(A) is a value between −2.0 and 0.5. In some embodiments, (A) is a value between −1.5 and 0.0. In some embodiments, (A) is a value between −1.0 and −0.5.

In some embodiments, (A) is a value between −1.5 and −0.4. In some embodiments, (A) is a value between −1.4 and −0.5. In some embodiments, (A) is a value between −1.3 and −0.6. In some embodiments, (A) is a value between −1.2 and −0.7. In some embodiments, (A) is a value between −1.1 and −0.8. In some embodiments, (A) is a value between −1.0 and −0.9.

In some embodiments, (A) is a value between −1.0 and 1.0. In some embodiments, (A) is a value between −0.9 and 0.9. In some embodiments, (A) is a value between −0.8 and 0.8. In some embodiments, (A) is a value between −0.7 and 0.7. In some embodiments, (A) is a value between −0.6 and 0.6. In some embodiments, (A) is a value between −0.5 and 0.5. In some embodiments, (A) is a value between −0.4 and 0.4. In some embodiments, (A) is a value between −0.3 and 0.3. In some embodiments, (A) is a value between −0.2 and 0.2. In some embodiments, (A) is a value between −0.1 and 0.1.

In some embodiments, (A) is about −2.0. In some embodiments, (A) is about −1.9. In some embodiments, (A) is about −1.8. In some embodiments, (A) is about −1.7. In some embodiments, (A) is about −1.6. In some embodiments, (A) is about −1.5. In some embodiments, (A) is about −1.4. In some embodiments, (A) is about −1.3. In some embodiments, (A) is about −1.2. In some embodiments, (A) is about −1.1. In some embodiments, (A) is about −1.0. In some embodiments, (A) is about −0.9. In some embodiments, (A) is about −0.8. In some embodiments, (A) is about −0.7. In some embodiments, (A) is about −0.6. In some embodiments, (A) is about −0.5. In some embodiments, (A) is about −0.4. In some embodiments, (A) is about −0.3. In some embodiments, (A) is about −0.2. In some embodiments, (A) is about −0.1. In some embodiments, (A) is about 0.1. In some embodiments, (A) is about 0.2. In some embodiments, (A) is about 0.3. In some embodiments, (A) is about 0.4. In some embodiments, (A) is about 0.5. In some embodiments, (A) is about 0.6. In some embodiments, (A) is about 0.7. In some embodiments, (A) is about 0.8. In some embodiments, (A) is about 0.9. In some embodiments, (A) is about 1.0.

In some embodiments, (A) is −0.95. In some embodiments, (A) is 0.02. In one embodiment, the cell type is DG44 and (A) is −0.95. In one embodiment, the cell type is CHOZN and (A) is 0.02.

In some embodiments, (B) is a value between −1.5 and 2.5. In some embodiments, (B) is a value between −1.0 and 2.0. In some embodiments, (B) is a value between −0.5 and 1.5. In some embodiments, (B) is a value between 0 and 1.0.

In some embodiments, (B) is a value between −0.5 and −0.4. In some embodiments, (B) is a value between −0.4 and −0.3. In some embodiments, (B) is a value between −0.3 and −0.2. In some embodiments, (B) is a value between −0.2 and −0.1. In some embodiments, (B) is a value between −0.1 and 0.0. In some embodiments, (B) is a value between 0.0 and 0.1. In some embodiments, (B) is a value between 0.1 and 0.2. In some embodiments, (B) is a value between 0.2 and 0.3. In some embodiments, (B) is a value between 0.3 and 0.4. In some embodiments, (B) is a value between 0.4 and 0.5. In some embodiments, (B) is a value between 0.5 and 0.6. In some embodiments, (B) is a value between 0.6 and 0.7. In some embodiments, (B) is a value between 0.7 and 0.8. In some embodiments, (B) is a value between 0.8 and 0.9. In some embodiments, (B) is a value between 0.9 and 1.0. In some embodiments, (B) is a value between 1.0 and 1.1. In some embodiments, (B) is a value between 1.1 and 1.2. In some embodiments, (B) is a value between 1.2 and 1.3. In some embodiments, (B) is a value between 1.3 and 1.4. In some embodiments, (B) is a value between 1.4 and 1.5.

In some embodiments, (B) is about −1.5. In some embodiments, (B) is about −1.4. In some embodiments, (B) is about −1.3. In some embodiments, (B) is about −1.2. In some embodiments, (B) is about −1.1. In some embodiments, (B) is about −1.0. In some embodiments, (B) is about −0.9. In some embodiments, (B) is about −0.8. In some embodiments, (B) is about −0.7. In some embodiments, (B) is about −0.6. In some embodiments, (B) is about −0.5. In some embodiments, (B) is about −0.4. In some embodiments, (B) is about −0.3. In some embodiments, (B) is about −0.2. In some embodiments, (B) is about −0.1. In some embodiments, (B) is about 0.1. In some embodiments, (B) is about 0.2. In some embodiments, (B) is about 0.3. In some embodiments, (B) is about 0.4. In some embodiments, (B) is about 0.5. In some embodiments, (B) is about 0.6. In some embodiments, (B) is about 0.7. In some embodiments, (B) is about 0.8. In some embodiments, (B) is about 0.9. In some embodiments, (B) is about 1.0. In some embodiments, (B) is about 1.1. In some embodiments, (B) is about 1.2. In some embodiments, (B) is about 1.3. In some embodiments, (B) is about 1.4. In some embodiments, (B) is about 1.5. In some embodiments, (B) is about 1.6. In some embodiments, (B) is about 1.7. In some embodiments, (B) is about 1.8. In some embodiments, (B) is about 1.9. In some embodiments, (B) is about 2.0.

In some embodiments, (B) is 0.86. In some embodiments, (B) is 0.13. In one embodiment, the cell type is DG44 and (B) is 0.86. In one embodiment, the cell type is CHOZN and (B) is 0.13.

In some embodiments, (C) is a value between 0 and 4.0. In some embodiments, (C) is a value between 0.5 and 3.5. In some embodiments, (C) is a value between 1.0 and 3.0. In some embodiments, (C) is a value between 1.5 and 2.5.

In some embodiments, (C) is a value between 0.0 and 0.1. In some embodiments,

(C) is a value between 0.1 and 0.2. In some embodiments, (C) is a value between 0.2 and 0.3. In some embodiments, (C) is a value between 0.3 and 0.4. In some embodiments, (C) is a value between 0.4 and 0.5. In some embodiments, (C) is a value between 0.5 and 0.6. In some embodiments, (C) is a value between 0.6 and 0.7. In some embodiments, (C) is a value between 0.7 and 0.8. In some embodiments, (C) is a value between 0.8 and 0.9. In some embodiments, (C) is a value between 0.9 and 1.0. In some embodiments, (C) is a value between 1.0 and 1.1. In some embodiments, (C) is a value between 1.1 and 1.2. In some embodiments, (C) is a value between 1.2 and 1.3. In some embodiments, (C) is a value between 1.3 and 1.4. In some embodiments, (C) is a value between 1.4 and 1.5. In some embodiments, (C) is a value between 1.5 and 1.6. In some embodiments, (C) is a value between 1.6 and 1.7. In some embodiments, (C) is a value between 1.7 and 1.8. In some embodiments, (C) is a value between 1.8 and 1.9. In some embodiments, (C) is a value between 1.9 and 2.0. In some embodiments, (C) is a value between 2.0 and 2.1. In some embodiments, (C) is a value between 2.1 and 2.2. In some embodiments, (C) is a value between 2.2 and 2.3. In some embodiments, (C) is a value between 2.3 and 2.4. In some embodiments, (C) is a value between 2.4 and 2.5. In some embodiments, (C) is a value between 2.5 and 2.6. In some embodiments, (C) is a value between 2.6 and 2.7. In some embodiments, (C) is a value between 2.7 and 2.8. In some embodiments, (C) is a value between 2.8 and 2.9. In some embodiments, (C) is a value between 2.9 and 3.0. In some embodiments, (C) is a value between 3.0 and 3.1. In some embodiments, (C) is a value between 3.1 and 3.2. In some embodiments, (C) is a value between 3.2 and 3.3. In some embodiments, (C) is a value between 3.3 and 3.4. In some embodiments, (C) is a value between 3.4 and 3.5. In some embodiments, (C) is a value between 3.5 and 3.6. In some embodiments, (C) is a value between 3.6 and 3.7. In some embodiments, (C) is a value between 3.7 and 3.8. In some embodiments, (C) is a value between 3.8 and 3.9. In some embodiments, (C) is a value between 3.9 and 4.0.

In some embodiments, (C) is 1.21. In some embodiments, (C) is 2.41. In one embodiment, the cell type is DG44 and (C) is 1.21. In one embodiment, the cell type is CHOZN and (C) is 2.41.

In some embodiments, A=−0.95, B=0.86, and C=1.21. In some embodiments, A=0.02, B=0.13, and C=2.41. In one embodiment, the cell type is DG44 and (A) is −0.95, (B) is 0.86, and (C) is 1.21. In one embodiment, the cell type is CHOZN, and (A) is 0.02, (B) is 0.13, and (C) is 2.41.

In some embodiments, disclosed herein is a method of increasing, controlling, or modulating protein yield in a sample mixture comprising a target protein and impurities comprising (a) flushing a harvest skid with water; (b) loading the sample into the harvest skid; (c) measuring an ultraviolet (UV) signal of the sample mixture during protein filtration in the harvest skid into a real-time protein titer; (d) starting collection of the protein based on the UV measurement and the real-time protein titer; (e) chasing the protein with PBS; and (f) stopping collection of the protein based on the UV measurement and the real-time protein titer; wherein the UV signal correlates with the real-time protein titer during the filtration.

In some embodiments, the methods described herein comprise a water (e.g., RODI) flush. In some embodiments, the methods comprise loading the protein sample and starting collection based on an online titer. In some embodiments, the methods comprise a PBS chase and an end collection based on an online titer. Compared with other methods, the methods disclosed herein do not comprise an air blow-down step.

In some embodiments, the start and end of collection of sample are controlled automatically based on online UV readings and calculated titer. In a particular embodiment, the real-time target protein concentration during the harvest process is calculated by online UV sensor readings, using modeling. In some embodiments, cut-off of bulk collection is determined directly on calculated online target protein concentration. In some embodiments, the calculation algorithms are integrated into Delta V™ control system to achieve automatic cut-off of protein collection.

In some embodiments, the methods disclosed herein comprise a step of modeling.

In some embodiments, modeling comprises offline titer measurements against online UV signals using serial dilution samples to establish a linear correlation between UV signal and titer. In some embodiments, the sample used in modeling is a purified protein. In some embodiments, the sample used in modeling is a bulk protein that comprises contaminants. In some embodiments, the modeling is then used to control, modulate, increase, and/or improve protein yield.

In some embodiments, the methods disclosed herein comprise controlling, modulating or improving yield of a target protein with a titer that is at least about 0.01 g/L. In some embodiments, the titer is at least about 0.02 g/L. In some embodiments, the titer is at least about 0.03 g/L. In some embodiments, the titer is at least about 0.04 g/L. In some embodiments, the titer is at least about 0.05 g/L. In some embodiments, the titer is at least about 0.06 g/L. In some embodiments, the titer is at least about 0.07 g/L. In some embodiments, the titer is at least about 0.08 g/L. In some embodiments, the titer is at least about 0.09 g/L. In some embodiments, the titer is at least about 0.1 g/L. In some embodiments, the titer is at least about 0.2 g/L. In some embodiments, the titer is at least about 0.3 g/L. In some embodiments, the titer is at least about 0.4 g/L. In some embodiments, the titer is at least about 0.5 g/L. In some embodiments, the titer is at least about 0.6 g/L. In some embodiments, the titer is at least about 0.7 g/L. In some embodiments, the titer is at least about 0.8 g/L. In some embodiments, the titer is at least about 0.9 g/L. In some embodiments, the titer is at least about 1 g/L. In some embodiments, the titer is at least about 1.5 g/L. In some embodiments, the titer is at least about 2 g/L. In some embodiments, the titer is at least about 2.5 g/L. In some embodiments, the titer is at least about 3 g/L. In some embodiments, the titer is at least about 3.5 g/L. In some embodiments, the titer is at least about 4 g/L. In some embodiments, the titer is at least about 4.5 g/L. In some embodiments, the titer is at least about 5 g/L. In some embodiments, the titer is at least about 5.5 g/L. In some embodiments, the titer is at least about 6 g/L. In some embodiments, the titer is at least about 6.5 g/L. In some embodiments, the titer is at least about 7 g/L. In some embodiments, the titer is at least about 7.5 g/L. In some embodiments, the titer is at least about 8 g/L. In some embodiments, the titer is at least about 8.5 g/L. In some embodiments, the titer is at least about 9 g/L. In some embodiments, the titer is at least about 9.5 g/L. In some embodiments, the titer is at least about 10 g/L. In some embodiments, the titer is at least about 10.5 g/L. In some embodiments, the titer is at least about 11 g/L. In some embodiments, the titer is at least about 11.5 g/L. In some embodiments, the titer is at least about 12 g/L. In some embodiments, the titer is at least about 12.5 g/L. In some embodiments, the titer is at least about 13 g/L. In some embodiments, the titer is at least about 13.5 g/L. In some embodiments, the titer is at least about 14 g/L. In some embodiments, the titer is at least about 14.5 g/L. In some embodiments, the titer is at least about 15 g/L. In some embodiments, the titer is at least about 15.5 g/L. In some embodiments, the titer is at least about 16 g/L. In some embodiments, the titer is at least about 16.5 g/L. In some embodiments, the titer is at least about 17 g/L. In some embodiments, the titer is at least about 17.5 g/L. In some embodiments, the titer is at least about 18 g/L, at least about 18.5 g/L. In some embodiments, the titer is at least about 19 g/L. In some embodiments, the titer is at least about 19.5 g/L. In some embodiments, the titer is at least about 20 g/L.

In some embodiments, the methods disclosed herein comprise collection of the target protein that is dependent on the titer of the target protein. In some embodiments, collection of the target protein begins when the titer is at least about 0.05 g/L. In some embodiments, collection of the target protein begins when the titer is at least about 0.06 g/L. In some embodiments, collection of the target protein begins when the titer is at least about 0.07 g/L. In some embodiments, collection of the target protein begins when the titer is at least about 0.08 g/L. In some embodiments, collection of the target protein begins when the titer is at least about 0.09 g/L. In some embodiments, collection of the target protein begins when the titer is at least about 0.1 g/L. In some embodiments, collection of the target protein begins when the titer is at least about 0.2 g/L. In some embodiments, collection of the target protein begins when the titer is at least about 0.3 g/L. In some embodiments, collection of the target protein begins when the titer is at least about 0.4 g/L. In some embodiments, collection of the target protein begins when the titer is at least about 0.5 g/L. In some embodiments, collection of the target protein begins when the titer is at least about 0.6 g/L. In some embodiments, collection of the target protein begins when the titer is at least about 0.7 g/L. In some embodiments, collection of the target protein begins when the titer is at least about 0.8 g/L. In some embodiments, collection of the target protein begins when the titer is at least about 0.9 g/L. In some embodiments, collection of the target protein begins when the titer is at least about 1 g/L. In some embodiments, collection of the target protein begins when the titer is at least about 1.5 g/L. In some embodiments, collection of the target protein begins when the titer is at least about 2 g/L. In some embodiments, collection of the target protein begins when the titer is at least about 2.5 g/L. In some embodiments, collection of the target protein begins when the titer is at least about 3 g/L. In some embodiments, collection of the target protein begins when the titer is at least about 3.5 g/L. In some embodiments, collection of the target protein begins when the titer is at least about 4 g/L. In some embodiments, collection of the target protein begins when the titer is at least about 4.5 g/L. In some embodiments, collection of the target protein begins when the titer is at least about 5 g/L. In some embodiments, collection of the target protein begins when the titer is at least about 5.5 g/L. In some embodiments, collection of the target protein begins when the titer is at least about 6 g/L. In some embodiments, collection of the target protein begins when the titer is at least about 6.5 g/L. In some embodiments, collection of the target protein begins when the titer is at least about 7 g/L. In some embodiments, collection of the target protein begins when the titer is at least about 7.5 g/L. In some embodiments, collection of the target protein begins when the titer is at least about 8 g/L. In some embodiments, collection of the target protein begins when the titer is at least about 8.5 g/L. In some embodiments, collection of the target protein begins when the titer is at least about 9 g/L. In some embodiments, collection of the target protein begins when the titer is at least about 9.5 g/L. In some embodiments, collection of the target protein begins when the titer is at least about 10 g/L. In some embodiments, collection of the target protein begins when the titer is at least about 10.5 g/L. In some embodiments, collection of the target protein begins when the titer is at least about 11 g/L. In some embodiments, collection of the target protein begins when the titer is at least about 11.5 g/L. In some embodiments, collection of the target protein begins when the titer is at least about 12 g/L. In some embodiments, collection of the target protein begins when the titer is at least about 12.5 g/L. In some embodiments, collection of the target protein begins when the titer is at least about 13 g/L. In some embodiments, collection of the target protein begins when the titer is at least about 13.5 g/L. In some embodiments, collection of the target protein begins when the titer is at least about 14 g/L. In some embodiments, collection of the target protein begins when the titer is at least about 14.5 g/L. In some embodiments, collection of the target protein begins when the titer is at least about 15 g/L. In some embodiments, collection of the target protein begins when the titer is at least about 15.5 g/L. In some embodiments, collection of the target protein begins when the titer is at least about 16 g/L. In some embodiments, collection of the target protein begins when the titer is at least about 16.5 g/L. In some embodiments, collection of the target protein begins when the titer is at least about 17 g/L. In some embodiments, collection of the target protein begins when the titer is at least about 17.5 g/L. In some embodiments, collection of the target protein begins when the titer is at least about 18 g/L. In some embodiments, collection of the target protein begins when the titer is at least about 18.5 g/L. In some embodiments, collection of the target protein begins when the titer is at least about 19 g/L. In some embodiments, collection of the target protein begins when the titer is at least about 19.5 g/L. In some embodiments, collection of the target protein begins when the titer is at least about 20 g/L.

In some embodiments, the methods disclosed herein comprise collection of a target protein, wherein the target protein has a titer in a range. In some embodiments, the titer at which the target protein is collected is between about 0.05 g/L and about 20 g/L. In some embodiments, the titer at which the target protein is collected is between about 0.1 g/L and about 20 g/L. In some embodiments, the titer at which the target protein is collected is between about 0.2 g/L and about 20 g/L. In some embodiments, the titer at which the target protein is collected is between about 0.3 g/L and about 20 g/L. In some embodiments, the titer at which the target protein is collected is between about 0.4 g/L and about 20 g/L. In some embodiments, the titer at which the target protein is collected is, between about 0.5 g/L and about 20 g/L. In some embodiments, the titer at which the target protein is collected is between about 0.6 g/L and about 20 g/L. In some embodiments, the titer at which the target protein is collected is between about 0.7 g/L and about 20 g/L. In some embodiments, the titer at which the target protein is collected is between about 0.8 g/L and about 20 g/L. In some embodiments, the titer at which the target protein is collected is between about 0.9 g/L and about 20 g/L. In some embodiments, the titer at which the target protein is collected is between about 1 g/L and about 20 g/L. In some embodiments, the titer at which the target protein is collected is between about 0.05 g/L and about 15 g/L. In some embodiments, the titer at which the target protein is collected is between about 0.1 g/L and about 15 g/L. In some embodiments, the titer at which the target protein is collected is between about 0.2 g/L and about 15 g/L. In some embodiments, the titer at which the target protein is collected is between about 0.3 g/L and about 15 g/L. In some embodiments, the titer at which the target protein is collected is between about 0.4 g/L and about 15 g/L. In some embodiments, the titer at which the target protein is collected is between about 0.5 g/L and about 15 g/L. In some embodiments, the titer at which the target protein is collected is between about 0.6 g/L and about 15 g/L. In some embodiments, the titer at which the target protein is collected is between about 0.7 g/L and about 15 g/L. In some embodiments, the titer at which the target protein is collected is between about 0.8 g/L and about 15 g/L. In some embodiments, the titer at which the target protein is collected is between about 0.9 g/L and about 15 g/L. In some embodiments, the titer at which the target protein is collected is between about 1 g/L and about 15 g/L. In some embodiments, the titer at which the target protein is collected is between about 0.05 g/L and about 10 g/L. In some embodiments, the titer at which the target protein is collected is between about 0.1 g/L and about 10 g/L. In some embodiments, the titer at which the target protein is collected is between about 0.2 g/L and about 10 g/L. In some embodiments, the titer at which the target protein is collected is between about 0.3 g/L and about 10 g/L. In some embodiments, the titer at which the target protein is collected is between about 0.4 g/L and about 10 g/L. In some embodiments, the titer at which the target protein is collected is between about 0.5 g/L and about 10 g/L. In some embodiments, the titer at which the target protein is collected is between about 0.6 g/L and about 10 g/L. In some embodiments, the titer at which the target protein is collected is between about 0.7 g/L and about 10 g/L. In some embodiments, the titer at which the target protein is collected is between about 0.8 g/L and about 10 g/L. In some embodiments, the titer at which the target protein is collected is between about 0.9 g/L and about 10 g/L. In some embodiments, the titer at which the target protein is collected is between about 1 g/L and about 10 g/L.

In some embodiments, the methods disclosed herein further comprise stopping the collection of the target protein when the collection titer is below about 0.5 g/L.

In some embodiments, the yield of the target protein is increased by the methods disclosed herein. In some embodiments, the target protein yield is increased at least about 1% compared to the protein yield without monitoring in real-time an ultraviolet (UV) signal of the sample mixture. In some embodiments, the target protein yield is increased at least about 2% compared to the protein yield without monitoring in real-time an ultraviolet (UV) signal of the sample mixture. In some embodiments, the target protein yield is increased at least about 3% compared to the protein yield without monitoring in real-time an ultraviolet (UV) signal of the sample mixture. In some embodiments, the target protein yield is increased at least about 4% compared to the protein yield without monitoring in real-time an ultraviolet (UV) signal of the sample mixture. In some embodiments, the target protein yield is increased at least about 5% compared to the protein yield without monitoring in real-time an ultraviolet (UV) signal of the sample mixture. In some embodiments, the target protein yield is increased at least about 6% compared to the protein yield without monitoring in real-time an ultraviolet (UV) signal of the sample mixture. In some embodiments, the target protein yield is increased at least about 7% compared to the protein yield without monitoring in real-time an ultraviolet (UV) signal of the sample mixture. In some embodiments, the target protein yield is increased at least about 8% compared to the protein yield without monitoring in real-time an ultraviolet (UV) signal of the sample mixture. In some embodiments, the target protein yield is increased at least about 9% compared to the protein yield without monitoring in real-time an ultraviolet (UV) signal of the sample mixture. In some embodiments, the target protein yield is increased at least about 10% compared to the protein yield without monitoring in real-time an ultraviolet (UV) signal of the sample mixture. In some embodiments, the target protein yield is increased at least about 11% compared to the protein yield without monitoring in real-time an ultraviolet (UV) signal of the sample mixture. In some embodiments, the target protein yield is increased at least about 12% compared to the protein yield without monitoring in real-time an ultraviolet (UV) signal of the sample mixture. In some embodiments, the target protein yield is increased at least about 13% compared to the protein yield without monitoring in real-time an ultraviolet (UV) signal of the sample mixture. In some embodiments, the target protein yield is increased at least about 14% compared to the protein yield without monitoring in real-time an ultraviolet (UV) signal of the sample mixture. In some embodiments, the target protein yield is increased at least about 15% compared to the protein yield without monitoring in real-time an ultraviolet (UV) signal of the sample mixture. In some embodiments, the target protein yield is increased at least about 16% compared to the protein yield without monitoring in real-time an ultraviolet (UV) signal of the sample mixture. In some embodiments, the target protein yield is increased at least about 17% compared to the protein yield without monitoring in real-time an ultraviolet (UV) signal of the sample mixture. In some embodiments, the target protein yield is increased at least about 18% compared to the protein yield without monitoring in real-time an ultraviolet (UV) signal of the sample mixture. In some embodiments, the target protein yield is increased at least about 19% compared to the protein yield without monitoring in real-time an ultraviolet (UV) signal of the sample mixture. In some embodiments, the target protein yield is increased or at least about 20% compared to the protein yield without monitoring in real-time an ultraviolet (UV) signal of the sample mixture.

In some embodiments, the ultraviolet (UV) signal of the sample mixture is measured from 0 to 2 AU. In other embodiments, the UV signal of the sample mixture is measured at about 0.1 AU, about 0.2 AU, about 0.3 AU, about 0.4 AU, about 0.5 AU, about 0.6 AU, about 0.7 AU, about 0.8 AU, about 0.9 AU, about 1.0 AU, about 1.1 AU, about 1.2 AU, about 1.3 AU, about 1.4 AU, about 1.5 AU, about 1.6 AU, about 1.7 AU, about 1.8 AU, about 1.9 AU, or about 2.0 AU.

In some embodiments disclosed herein, the methods comprise protein filtration. In some embodiments, the methods comprises one or more filters. In some embodiments, the protein filtration is a depth filtration. In some embodiments, the depth filtration comprises a primary depth filter and a secondary depth filter. In some embodiments, the depth filtration comprises a primary depth filter.

In some embodiments, the methods comprise loading the sample mixture prior to the monitoring.

In some embodiments, the methods comprise flushing the depth filters with a buffer before loading the cell culture and chasing the depth filters post loading the cell culture. In some embodiments, the methods comprise chasing the sample mixture with phosphate buffered saline (PBS). In some embodiments, the methods comprise a harvest skid which comprises a control system wherein the control system automatically starts collection of the protein when the titer is above 0.5 g/L. In some embodiments, the methods comprise a harvest skid comprises a control system, wherein the control system automatically stops collection of the protein when the titer is below 0.5 g/L.

In some embodiments, the methods comprise a control system modulates flow rate of a liquid through the harvest skid. In some embodiments, the methods comprise a control system that automatically drives the pump to up-regulate flow rate through the harvest skid. In some embodiments, the methods comprise a control system that automatically drives the pump to down-regulate flow rate through the harvest skid. In some embodiments, the methods do not comprise a step of air blow-down.

In some embodiments, the methods comprise a step of collecting a protein yield that is not based on volume.

In some embodiments, the methods disclosed herein comprise measuring pressure, turbidity, temperature, flow rate, or any combination thereof.

In some embodiments, the methods comprise measuring pressure using a pressure sensor. In some embodiments, the pressure is measured in a range of −10 pounds per square inch (psi) to 50 psi, −10 psi to 40 psi, −9 psi to 40 psi, −8 psi to 40 psi, −7 psi to 30 psi, −6 psi to −20 psi, −7 psi to 40 psi, −8 psi to 40 psi, −9 psi to 45 psi, −10 psi to −45 psi, or −7 psi to −45 psi. In other embodiments, the pressure can be measured at least once, twice, three times, four times, or five times, e.g., before the primary filter, after the primary filter and before the secondary filter, after the secondary filter, after drain, or any combination thereof.

In some embodiments, the methods comprise measuring turbidity. In some embodiments, the turbidity is measured in a range of 0 absorbance units (AU) to 2 AU. In other embodiments, the turbidity is measured at about 0.1 AU, about 0.2 AU, about 0.3 AU, about 0.4 AU, about 0.5 AU, about 0.6 AU, about 0.7 AU, about 0.8 AU, about 0.9 AU, about 1.0 AU, about 1.1 AU, about 1.2 AU, about 1.3 AU, about 1.4 AU, about 1.5 AU, about 1.6 AU, about 1.7 AU, about 1.8 AU, about 1.9 AU, or about 2.0 AU. In some embodiments, the turbidity is measured at least once, twice, three times, four times, or five times, e.g., after the primary filter, after the secondary filter, or after the primary filter and the secondary filter. See FIG. 2.

In some embodiments, the methods comprise measuring temperature. In some embodiments, the temperature is measured in a range of 0° C. to 70° C., 0° C. to 60° C., 0° C. to 50° C., 0° C. to 40° C., 5° C. to 70° C., 10° C. to 70° C., 15° C. to 70° C., 20° C. to 70° C., 10° C. to 60° C., 20° C. to 50° C., 20° C. to 40° C., 20° C. to 45° C., 30° C. to 40° C., 35° C. to 40° C., 20° C. to 30° C., 35° C. to 40° C., or 25° C. to 45° C. In other embodiments, the temperature can be measured any time during the filtration process, e.g., at least once, twice, three times, four times, or five times, e.g., after the primary filter, after the secondary filter, or after the primary and the secondary filters. See FIG. 2.

In some embodiments, the methods comprise measuring flow. In some embodiments, the flow is measured in a range of 0 L/min to 20 L/min, 0 L/min to 30 L/min, 0 L/min to 40 L/min, 0 L/min to 50 L/min, 0 L/min to 60 L/min, 0 L/min to 70 L/min, 0 L/min to 80 L/min, 0 L/min to 90 L/min, 0 L/min to 100 L/min, 0 L/min to 110 L/min, 0 L/min to 120 L/min, 0 L/min to 130 L/min, 0 L/min to 140 L/min, 0 L/min to 150 L/min, 0 L/min to 160 L/min, 0 L/min to 170 L/min, 0 L/min to 180 L/min, 0 L/min to 190 L/min, 0 L/min to 200 L/min, 0 L/min to 250 L/min, or 0 L/min to 300 L/min. In other embodiments, the flow is measured any time during the filtration process: before the primary filter, after the primary filter, before the secondary filter, after the secondary filter, or any combination thereof.

In some embodiments, liquid from water source/bioreactor/PBS source is driven to primary depth filter by the LEVITRONIX® gravity pump.

In some embodiments, a system, e.g., Delta V™, can be employed to calculate flow totalizer volume through online flow sensor readings. In some embodiments flow totalizer volume is used to determine the end of water flush. In some embodiments, four pressure sensors are placed before primary depth filter, secondary depth filter, pre-filter and sterile filter. Pressure-flow control loop can work based on real-time pressure value before the primary depth filter. If the pressure values exceed a certain threshold, Delta V™ automatically drives the pump to down-regulate flow rate. In some embodiments, two turbidity sensors are placed after primary and secondary depth filters as indicators of filtrate quality. In some embodiments, one UV sensor, value of which is used to calculate online target protein concentration and control cut-off of clarified bulk collection, is placed after the secondary depth filter. Real-time upstream source and downstream receiving vessel weights were monitored and displayed on Delta V™ as well. In some embodiments, weight is monitors from 0 to 550 kg, with a measurement accuracy of 0.01 kg.

In some embodiments, protein is isolated from a source. In some embodiments, the sample mixture is selected from the group consisting of a pure protein sample, a clarified bulk protein sample, a cell culture sample, and any combination thereof. In some embodiments, the source is selected from cultured cells.

In some embodiments, the cells are prokaryotes. In bacterial systems, a number of expression vectors can be advantageously selected depending upon the use intended for the protein molecule being expressed. For example, when a large quantity of such a protein is to be produced, for the generation of pharmaceutical compositions of a protein molecule, vectors which direct the expression of high levels of protein products that are readily purified can be desirable.

In other embodiments, the cells are eukaryotes. In some embodiments, the cells are mammalian cells. In some embodiments, the cells are selected from Chinese hamster ovary (CHO) cells, HEK293 cells, mouse myeloma (NS0), baby hamster kidney cells (BHK), monkey kidney fibroblast cells (COS-7), Madin-Darby bovine kidney cells (MDBK), and any combination thereof. In one embodiment, the cells are Chinese hamster ovary cells. In some embodiments, the cells are insect cells, e.g., Spodoptera frugiperda cells.

In other embodiments, the cells are mammalian cells. Such mammalian cells include but are not limited to CHO, VERO, BHK, Hela, MDCK, HEK 293, NIH 3T3, W138, BT483, Hs578T, HTB2, BT2O and T47D, NSO, CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

In some embodiments, the mammalian cells are CHO cells. In some embodiments the CHO cell is CHO-DG44, CHOZN, CHO/dhfr-, CHOK1SV GS-KO, or CHO-S. In some embodiments, the CHO cell is CHO-DG4. In some embodiments, the CHO cell is CHOZN.

Other suitable CHO cell lines disclosed herein include CHO-K (e.g., CHO K1), CHO pro3-, CHO P12, CHO-K1/SF, DUXB11, CHO DUKX; PA-DUKX; CHO pro5; DUK-BII or derivatives thereof.

In some embodiments, the target protein is harvested from a culture medium having a cell density of at least about 1×10⁶ cells/mL. In some embodiments, the target protein is harvested from a culture medium having a cell density of at least about 5×10⁶ cells/mL. In some embodiments, the target protein is harvested from a culture medium having a cell density of at least about 1×10⁷ cells/mL. In some embodiments, the target protein is harvested from a culture medium having a cell density of at least about 1.5×10⁷ cells/mL. In some embodiments, the target protein is harvested from a culture medium having a cell density of at least about 2×10⁷ cells/mL. In some embodiments, the target protein is harvested from a culture medium having a cell density of at least about 2.5×10⁷ cells/mL. In some embodiments, the target protein is harvested from a culture medium having a cell density of at least about 3×10⁷ cells/mL. In some embodiments, the target protein is harvested from a culture medium having a cell density of at least about 3.5×10⁷ cells/mL. In some embodiments, the target protein is harvested from a culture medium having a cell density of at least about 4×10⁷ cells/mL. In some embodiments, the target protein is harvested from a culture medium having a cell density of at least about 4.5×10⁷ cells/mL. In some embodiments, the target protein is harvested from a culture medium having a cell density of at least about 5×10⁷ cells/mL.

In some embodiments, the source of the protein is bulk protein. In some embodiments, the source of the protein is a composition comprising protein and non-protein components. The non-protein components can include DNA and other contaminant.

In some embodiments the source of the protein is from an animal. In some embodiments, the animal is a mammal such as a non-primate (e.g., cow, pig, horse, cat, dog, rat etc.) or a primate (e.g., monkey or human). In some embodiments, the source is a tissue or cells from a human. In certain embodiments, such terms refer to a non-human animal (e.g., a non-human animal such as a pig, horse, cow, cat or dog). In some embodiments, such terms refer to a pet or farm animal. In specific embodiments, such terms refer to a human.

In some embodiments, the proteins purified by the methods described herein are fusion proteins. A “fusion” or “fusion” protein comprises a first amino acid sequence linked in frame to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide. A fusion protein is created, for example, by chemical synthesis, or by creating and translating a polynucleotide in which the peptide regions are encoded in the desired relationship. A fusion protein can further comprise a second amino acid sequence associated with the first amino acid sequence by a covalent, non-peptide bond or a non-covalent bond. Upon transcription/translation, a single protein is made. In this way, multiple proteins, or fragments thereof can be incorporated into a single polypeptide. “Operably linked” is intended to mean a functional linkage between two or more elements. For example, an operable linkage between two polypeptides fuses both polypeptides together in frame to produce a single polypeptide fusion protein. In a particular aspect, the fusion protein further comprises a third polypeptide which, as discussed in further detail below, can comprise a linker sequence.

In some embodiments, the proteins purified by the methods described herein are antibodies. Antibodies can include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab')₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody, preferably that is an immunoglobulin. In certain embodiments, an antibody described herein is an IgG₁, or IgG₄ antibody.

In some embodiments, the protein described herein is an “antigen-binding domain,” “antigen-binding region,” “antigen-binding fragment,” and similar terms, which refer to a portion of an antibody molecule which comprises the amino acid residues that confer on the antibody molecule its specificity for the antigen (e.g., the complementarity determining regions (CDR)). The antigen-binding region can be derived from any animal species, such as rodents (e.g., mouse, rat or hamster) and humans.

In some embodiments, the protein is an anti-LAG3 antibody, an anti-CTLA-4 antibody, an anti-TIM3 antibody, an anti-NKG2a antibody, an anti-ICOS antibody, an anti-CD137 antibody, an anti-KIR antibody, an anti-TGFβ antibody, an anti-IL-10 antibody, an anti-B7-H4 antibody, an anti-Fas ligand antibody, an anti-mesothelin antibody, an anti-CD27 antibody, an anti-GITR antibody, an anti-CXCR4 antibody, an anti-CD73 antibody, an anti-TIGIT antibody, an anti-OX40 antibody, an anti-PD-1 antibody, an anti-PD-L1 antibody, an anti-IL8 antibody, or any combination thereof. In some embodiments, the protein is Abatacept NGP. In other embodiments, the protein is Belatacept NGP.

In some embodiments, the protein is an anti-GITR (glucocorticoid-induced tumor necrosis factor receptor family-related gene) antibody. In some embodiments, the anti-GITR antibody has the CDR sequences of 6C8, e.g., a humanized antibody having the CDRs of 6C8, as described, e.g., in WO2006/105021; an antibody comprising the CDRs of an anti-GITR antibody described in WO2011/028683; an antibody comprising the CDRs of an anti-GITR antibody described in JP2008278814, an antibody comprising the CDRs of an anti-GITR antibody described in WO2015/031667, WO2015/187835, WO2015/184099, WO2016/054638, WO2016/057841, WO2016/057846, WO 2018/013818, or other anti-GITR antibody described or referred to herein, all of which are incorporated herein in their entireties.

In other embodiments, the protein is an anti-LAG3 antibody. Lymphocyte-activation gene 3, also known as LAG-3, is a protein which in humans is encoded by the LAG3 gene. LAG3, which was discovered in 1990 and is a cell surface molecule with diverse biologic effects on T cell function. It is an immune checkpoint receptor and as such is the target of various drug development programs by pharmaceutical companies seeking to develop new treatments for cancer and autoimmune disorders. In soluble form it is also being developed as a cancer drug in its own right. Examples of anti-LAG3 antibodies include, but are not limited to, the antibodies in WO 2017/087901 A2, WO 2016/028672 A1, WO 2017/106129 A1, WO 2017/198741 A1, US 2017/0097333 A1, US 2017/0290914 A1, and US 2017/0267759 A1, all of which are incorporated herein in their entireties.

In some embodiments, the protein is an anti-CXCR4 antibody. CXCR4 is a 7 transmembrane protein, coupled to G1. CXCR4 is widely expressed on cells of hemopoietic origin, and is a major co-receptor with CD4+for human immunodeficiency virus 1 (HIV-1) See Feng, Y., Broeder, C. C., Kennedy, P. E., and Berger, E. A. (1996) Science 272, 872-877. Examples of anti-CXCR4 antibodies include, but are not limited to, the antibodies in WO 2009/140124 A1, US 2014/0286936 A1, WO 2010/125162 A1, WO 2012/047339 A2, WO 2013/013025 A2, WO 2015/069874 A1, WO 2008/142303 A2, WO 2011/121040 A1, WO 2011/154580 A1, WO 2013/071068 A2, and WO 2012/175576 A1, all of which are incorporated herein in their entireties.

In some embodiments, the protein is an anti-CD73 (ecto-5′-nucleotidase) antibody. In some embodiments, the anti-CD73 antibody inhibits the formation of adenosine. Degradation of AMP into adenosine results in the generation of an immunosuppressed and pro-angiogenic niche within the tumor microenvironment that promotes the onset and progression of cancer. Examples of anti-CD73 antibodies include, but are not limited to, the antibodies in WO 2017/100670 A1, WO 2018/013611 A1, WO 2017/152085 A1, and WO 2016/075176 A1, all of which are incorporated herein in their entireties.

In some embodiments, the protein is an anti-TIGIT (T cell Immunoreceptor with Ig and ITIM domains) antibody. TIGIT is a member of the PVR (poliovirus receptor) family of immunoglobin proteins. TIGIT is expressed on several classes of T cells including follicular B helper T cells (TFH). The protein has been shown to bind PVR with high affinity; this binding is thought to assist interactions between TFH and dendritic cells to regulate T cell dependent B cell responses. Examples of anti-TIGIT antibodies include, but are not limited to, the antibodies in WO 2016/028656 A1, WO 2017/030823 A2, WO 2017/053748 A2, WO 2018/033798 A1, WO 2017/059095 A1, and WO 2016/011264 A1, all of which are incorporated herein by their entireties.

In some embodiments, the protein is an anti-OX40 (i.e., CD134) antibody. 0X40 is a cytokine of the tumor necrosis factor (TNF) ligand family. OX40 functions in T cell antigen-presenting cell (APC) interactions and mediates adhesion of activated T cells to endothelial cells. Examples of anti-OX40 antibodies include, but are not limited to, WO 2018/031490 A2, WO 2015/153513 A1, WO 2017/021912 A1, WO 2017/050729 A1, WO 2017/096182 A1, WO 2017/134292 A1, WO 2013/038191 A2, WO 2017/096281 A1, WO 2013/028231 A1, WO 2016/057667 A1, WO 2014/148895 A1, WO 2016/200836 A1, WO 2016/100929 A1, WO 2015/153514 A1, WO 2016/002820 A1, and WO 2016/200835 A1, all of which are incorporated herein by their entireties.

In some embodiments, the protein is an anti-IL8 antibody. IL-8 is a chemotactic factor that attracts neutrophils, basophils, and T-cells, but not monocytes. It is also involved in neutrophil activation. It is released from several cell types in response to an inflammatory stimulus.

In some embodiments, the protein is Abatacept (marketed as ORENCIA®). Abatacept (also abbreviated herein as Aba) is a drug used to treat autoimmune diseases like rheumatoid arthritis, by interfering with the immune activity of T cells. Abatacept is a fusion protein composed of the Fc region of the immunoglobulin IgG1 fused to the extracellular domain of CTLA-4. In order for a T cell to be activated and produce an immune response, an antigen presenting cell must present two signals to the T cell. One of those signals is the major histocompatibility complex (MHC), combined with the antigen, and the other signal is the CD80 or CD86 molecule (also known as B7-1 and B7-2).

In some embodiments, the protein is Belatacept (trade name NULOJIX®). Belatacept is a fusion protein composed of the Fc fragment of a human IgG1 immunoglobulin linked to the extracellular domain of CTLA-4, which is a molecule crucial in the regulation of T cell costimulation, selectively blocking the process of T-cell activation. It is intended to provide extended graft and transplant survival while limiting the toxicity generated by standard immune suppressing regimens, such as calcineurin inhibitors. It differs from abatacept (ORENCIA®) by only 2 amino acids.
c. Systems

In some embodiments, disclosed herein is a system for controlling, modulating, increasing, or improving protein yield in a sample mixture comprising a target protein and impurities comprising monitoring in real-time an ultraviolet (UV) signal of the sample mixture during protein filtration in a harvest skid.

Systems disclosed herein comprise one or more sensors. In some embodiments, the sensors comprise pressure sensors, UV sensors, turbidity sensors, temperature sensors, flow sensors, and any combination thereof.

In some embodiments, the harvest skid is designed to integrate all sensors, including pressure (4), UV (1), turbidity (2), temperature (2) and flow sensor (1), into one cart. In some embodiments, the system comprises three PMAT (Pressure Monitor Alarm Transmitter) controllers. In some embodiments, the PMAT controllers are built on the cart to accommodate a total of ten different sensors. In some embodiments, a gravity pump (e.g., a LEVITRONIX® gravity pump) is used to drive liquid to depth filters and is mounted on the skid cart. In some embodiment, the system is movable, lockable and/or e-stoppable.

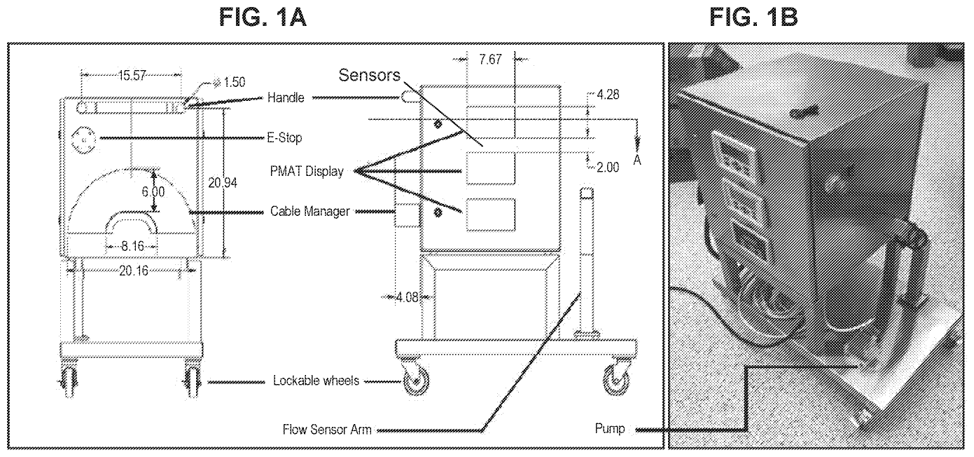

Also provided herein are systems (e.g., devices, e.g., harvest skid) that can be used in the above methods. In one embodiment, a system or device comprises the embodiments in FIG. 1a and/or FIG. 1b. In one embodiment, a system or device comprises the embodiment of FIG. 2.

In some embodiments, disclosed herein is an apparatus for controlling, modulating, increasing, or improving protein yield in a sample mixture comprising a target protein and impurities. The apparatus may include one or more sensor. The sensors may comprise pressure sensors, UV sensors, turbidity sensors, temperature sensors, flow sensors, and any combination thereof.

In some embodiments, the apparatus is designed to integrate all sensors, including pressure (4), UV (1), turbidity (2), temperature (2) and flow sensor (1), into the apparatus. In some embodiments, the apparatus comprises three PMAT (Pressure Monitor Alarm Transmitter) controllers. In some embodiments, the PMAT controllers are built into the apparatus to accommodate a total of ten different sensors. In some embodiments, a gravity pump (e.g., a LEVITRONIX® gravity pump) is used to drive liquid to depth filters and is mounted in the apparatus. In some embodiments, the system is movable, lockable and/or e-stoppable. The apparatus may also comprise a processor configured to control collection of the target protein. The processor may also be configured to change a condition of the apparatus, for example, the temperature, pressure, turbidity, or flow. The processor may also be configured to control the collection of the target protein. In some embodiments, the processor may use an established model to determine a culture harvesting process. The cell culture harvesting process may comprise a filtration based cell culture harvesting process. The processor may be configured to use a target protein titer. The apparatus may be incorporate into a system for controlling, modulating, increasing, or improving protein yield in a sample mixture comprising a target protein and impurities.
d. Process

In one embodiment, a system or device comprises the embodiment of FIG. 2., which demonstrates the process flow using this harvest skid. Liquid from water source/bioreactor/PBS source was driven to depth filter by the LEVITRONIX® gravity pump. A LEVITRONIX® flow sensor was placed after the pump. Delta V™ calculated flow totalizer volume through online flow sensor readings. Flow totalizer volume was used to determine the end of water flush. Four pressure sensors were placed before primary depth filter, secondary depth filter, pre-filter and sterile filter. Pressure-flow control loop worked based on real-time pressure value before the primary depth filter. Two turbidity sensors were placed after primary and secondary depth filters as indicators of filtrate quality. One UV sensor was placed after the secondary depth filter and used to calculate online target protein concentration and control cut-off of clarified bulk collection. Real-time upstream source and downstream receiving vessel weights were monitored and displayed on Delta V™ as well.

The following examples are offered by way of illustration and not by way of limitation.

### EXAMPLES

### Example 1

### Harvest Skid Design

In order to control, modulate, increase or improve protein yield in a sample, a harvest skid was utilized. FIG. 1 shows a schematic of the harvest skid. This harvest skid was designed to integrate all sensors, including pressure (4), UV (1), turbidity (2), temperature (2) and flow sensor (1), into one cart. See FIG. 2. Three PMAT controllers were built on the cart to accommodate a total of ten different sensors. A LEVITRONIX® gravity pump, which was used to drive liquid to depth filters, was also mounted on the skid cart. This harvest skid was designed to be movable, lockable and e-stoppable.

TABLE 1
instruments were used to design the skid

| Instrument/Material | Model | Vendor | Number |
|---|---|---|---|
| Pressure Sensor | SPECSS-N-ADJ-M | PENDOTECH ® | 4 |
| UV Sensor | SPECSS-N-ADJ-M | PENDOTECH ® | 1 |
| Turbidity Sensor | SPECPS-N-050 | PENDOTECH ® | 2 |
| Temperature Sensor | CONDS-N-050 | PENDOTECH ® | 2 |
| Pump | SEV11ATEX0121X | LEVITRONIX ® | 1 |
| Flow Sensor | LFS-10SU-Z | LEVITRONIX ® | 1 |
| Cart | Custom Design | High Purity New | 1 |
| | | England | |

TABLE 2
Instrument and material used in harvest process

| Instrument/Material | Vendor | Cat. No. |
|---|---|---|
| Primary depth filter | 3M | E16E07A10SP02A |
| Secondary depth filter | 3M | E16E01A90ZB05A |
| Pre-filter | Sartorius | 5478307F1G-SS |
| Sterile filter | Sartorius | 5447307H1--SS |
| PBS | NA | Made in house |
| Xcellerex Bioreactor | GE | 29119163 |
| Single use mixer | Thermo Fisher | SUM0500.0844 |

The sensors used by the harvest have distinct functions. The pressure sensor monitors pressure during process; cascade control to inlet pump, reduce inlet pump flow rate when pressure is too high. The UV sensor monitors UV signal after depth filtration during process; translated to protein concentration to control start and end of bulk collection. UV is measures at 280 nm.

The weight sensor monitors upstream bioreactor and downstream receiver weight during process; control load and chase steps. Bioreactor and receiver load cell values are integrated into harvest skid control system. The turbidity sensor, which measures turbidity at 880 nm, monitors turbidity before and after depth filtration during process. Turbidity breakthrough is observed if the depth filters are fouled. The temperature sensor monitors temperature during process. The harvest process herein operates under ambient (room) temperature.

The harvest skid process was used to purify proteins of interest from cell culture. Liquid from water source/bioreactor/PBS source was driven to primary depth filter by the LEVITRONIX® gravity pump. LEVITRONIX® gravity pump was proven to cause less cell death in CHO cell culture than the peristaltic pumps P3P. A LEVITRONIX® flow sensor was placed after the pump. Delta V™ calculated flow totalizer volume through online flow sensor readings. Flow totalizer volume was used to determine the end of water flush. Four pressure sensors were placed before primary depth filter, secondary depth filter, pre-filter and sterile filter P4P. Pressure-flow control loop worked based on real-time pressure value before the primary depth filter. If the pressure values exceed a certain threshold, Delta V™ would automatically drive the pump to down-regulate pump speed. Two turbidity sensors were placed after primary and secondary depth filters as indicators of filtrate quality. One UV sensor, value of which was used to calculate online target protein concentration and control cut-off of clarified bulk collection, was placed after the secondary depth filter. Real-time upstream source and downstream receiving vessel weights were monitored and displayed on Delta V™ as well.

For each of the examples disclosed herein, the harvest skid uses each sensor to detect values at the following ranges and accuracies.

TABLE 3

| | | Measurement | Measurement |
|---|---|---|---|
| Sensor | Role | Range | Accuracy |
| Pressure | Monitor and | −7 to 30 psi | Less than 0.9 psi |
| | Control | | |
| Flow | Monitor and | 0 to 20 L/min | Less than 0.18 L/min |
| | Control | | |
| UV | Monitor and | 0 to 2 AU | 0.02 AU |
| | Control | | |
| Weight | Monitor and | 0 to 550 kg | 0.01 kg |
| | Control | | |
| Turbidity | Monitor | 0 to 2 AU | 0.02 AU |
| Temperature | Monitor | 0 to 70° C. | 0.2° C. |

### Example 2

### Translating UV Signal to Protein Concentration

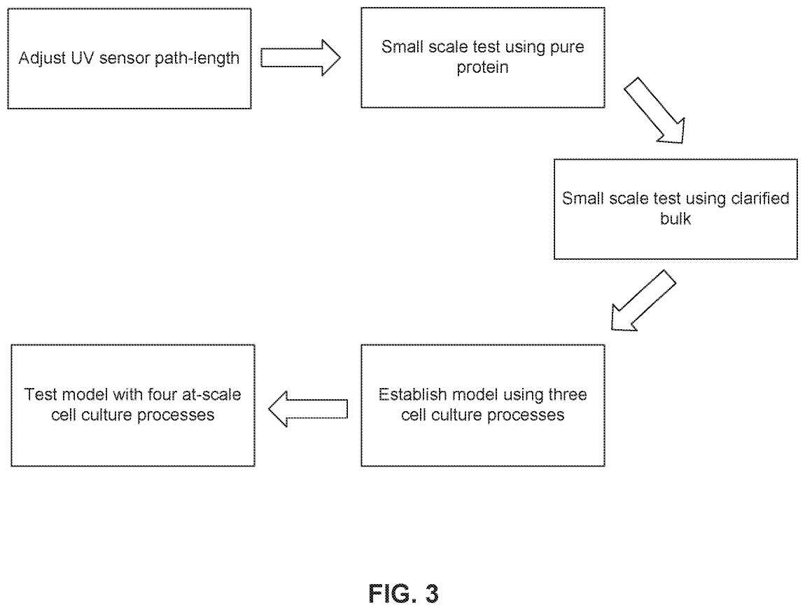

Compared with previous methods, the methods disclosed herein eliminated air blow-down step. Meanwhile, the start and end of collection of clarified bulk were controlled automatically based on online UV readings and calculated titer. See FIG. 3. More specifically, real-time target protein concentration during the harvest process was calculated by online UV sensor readings, using the models generated. Cut-off of bulk collection was therefore determined directly on calculated online target protein concentration. The calculation algorithms were integrated into Delta V™ control system to achieve automatic cut-off of clarified bulk collection.

The online UV sensor used in this harvest skid had an output absorbance from 0-2 AU. Path-length of this UV sensor was adjusted to accommodate a total target protein concentration of 0-6 g/L in the range of 0-2 AU. Other UV sensors or Flow VPE (C-technologies) could be used for higher concentration determination.

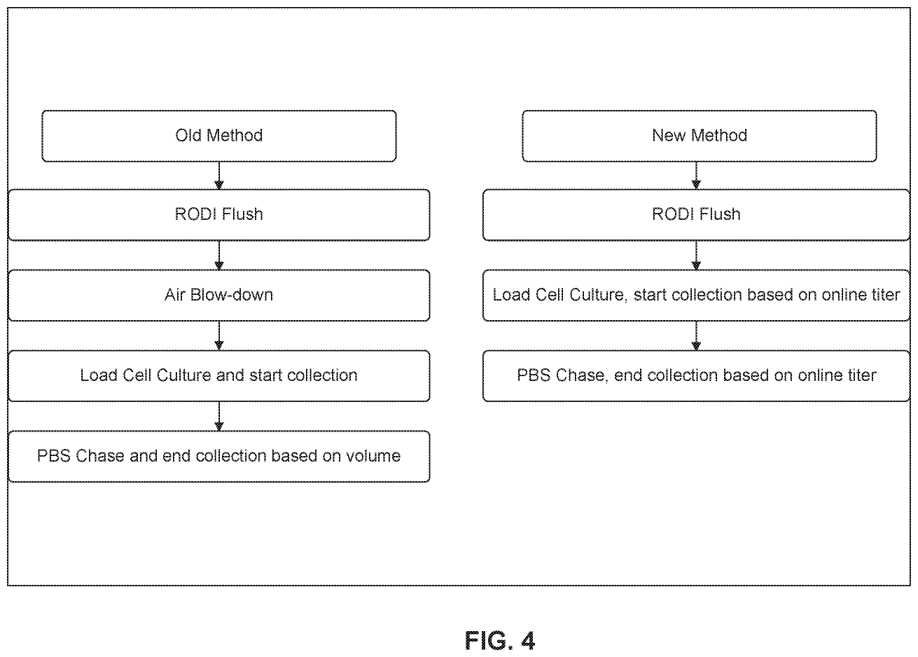

To translate online UV signal to target protein concentration in process, a sequential series of steps were taken as shown in FIG. 4.

In the first step, offline titer measurements against online UV signals using serial dilution samples of D12 GITR cell culture were determined. Several components in the cell culture sample, including target protein, HCP (host cell protein) and media pigments, could contribute to UV absorbance signal. To mimic the real life harvest process (where UV sensor measures total absorbance from all these components), a cell culture sample (D12 GITR cell culture), instead of a pure protein, was serially diluted and used for UV sensor path-length adjustment.

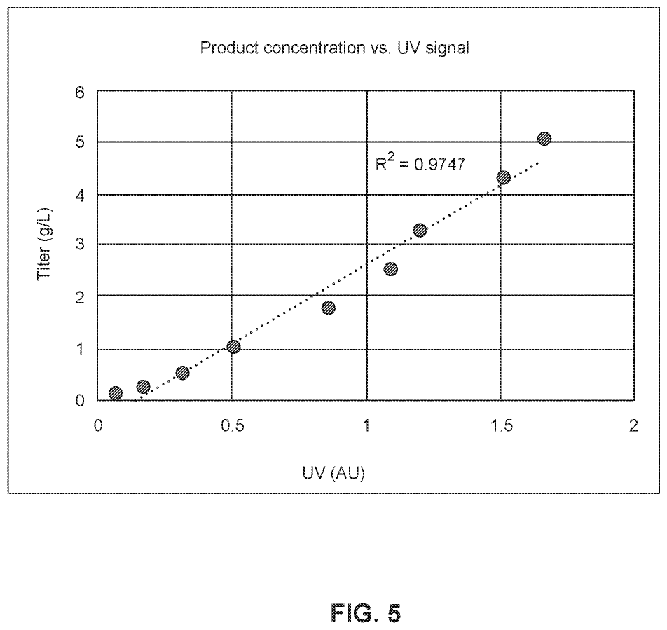

As shown in FIG. 5, UV sensor path length was adjusted to cover a wide range of target protein concentrations that could be observed during the harvest process. As UV readings close to 2 AU (maximum output) are less accurate, path length was adjusted down so that UV reading was around 1.6 at titer of 5 g/L. Good linearity was observed with R² of 0.97. Thus, the serial dilution of a cell culture sample provides a strong correlation between UV reading and titer.

### Example 3

### Small Scale Test using Pure Protein and Clarified Bulk

A small scale test was conducted using 2 L of pure protein (eTau) with a titer of 5.2 g/L. The depth filters were scaled down based on a loading capacity of 60 L/m² (per primary filter). Offline samples after secondary depth filter were collected during the harvest process. Offline titer readings were plotted with online UV sensor values to understand the relationship between pure protein concentration and online UV signal during harvest process.

A second small scale harvest test was carried out using 2 L of clarified bulk (eTau cell culture with cells removed) with a titer of 5 g/L. The depth filters were scaled down based on a loading capacity of 60 L/m² (per primary filter). Offline samples after secondary depth filter were collected during the harvest process. Offline titer readings were plotted with online UV sensor values to understand the relationship between target protein concentration (in a mixture of culture components) and online UV signal during harvest process.

Online UV and offline titer values during the test harvest process were plotted in

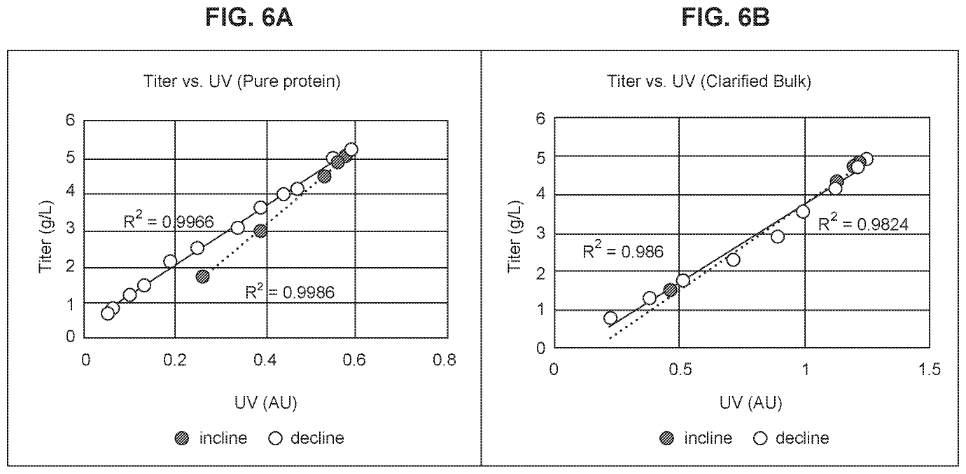

FIGS. 6a and 6b. The data series for “incline” were collected from start of bulk collection to end of loading; while the data series for “decline” were collected from start of chasing to end of bulk collection. As shown in FIGS. 6a and 6b, good linearity was observed for both processes for both incline and decline portion of the data. Thus, the serial dilution of a cell culture sample provides a strong correlation between UV reading and titer when measure other samples (e.g., pure protein in FIG. 6a and clarified bulk protein in FIG. 6b).

However, the slopes were different for the incline and decline portions, suggesting that separate models might be needed for different stages in harvest process.

### Example 4

### Establish Model Using Three Large Scale Cell Culture Processes

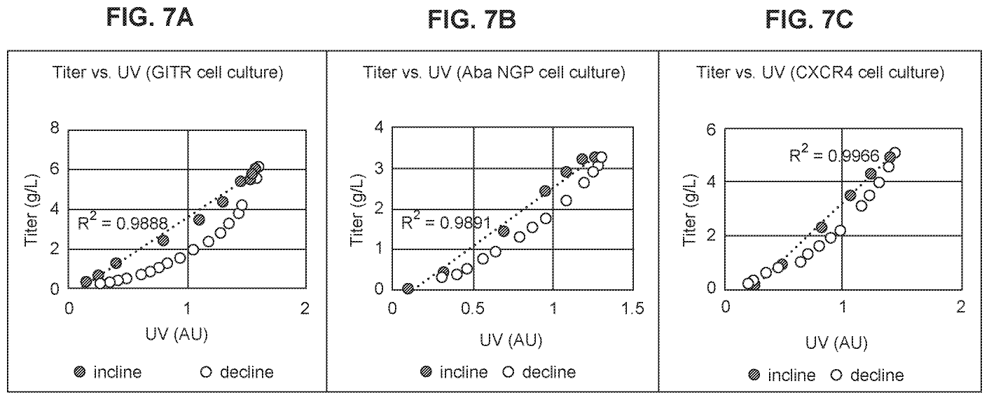

Three different cell lines were used for model establishment. These cell lines comprised different, large scale cell culture processes (Aba NGP, GITR and Next Gen CXCR4) with different characteristics (cell density, viability, titer, background noise, etc.). The cell lines were harvested using this harvest skid to generate data for model establishment. The depth filters were scaled based on a loading capacity of 60-65 L/m² (per primary filter). Offline samples after secondary depth filter were collected during the harvest processes. Offline titer readings and corresponding online UV sensor values were entered into JMP software to generate models. UV and titer values were plotted in FIG. 7a, FIG. 7b, and FIG. 7c. Good linearity was still observed for incline portion of the data. However, for the decline portion, curvature was observed.

During loading of cell culture material, cells, target protein, background noise protein all occupied depth filter membrane space. When PBS chase started, target protein were washed out. As PBS chase proceeded, background noise protein such as HCP that were loosely bound to depth filter membrane started to be washed out with target protein. At the same time, a release of HCP from cell debris also contributed to an increase in background noise percentage PSP. This might be the reason for the difference in UV profiles between clarified bulk sample and cell culture sample. In other words, for complex cell containing material, target protein contributed to total UV signal less and less, with background noise increasing, as PBS chase proceeded.

Based on these results, two separate models were established to predict target protein concentration using online UV signal: one is a linear model to fit the data from incline portion (start collection to end loading) and the other is a non-linear model to fit the data from decline portion (start chasing to end collection).

A. Model fit for incline portion.

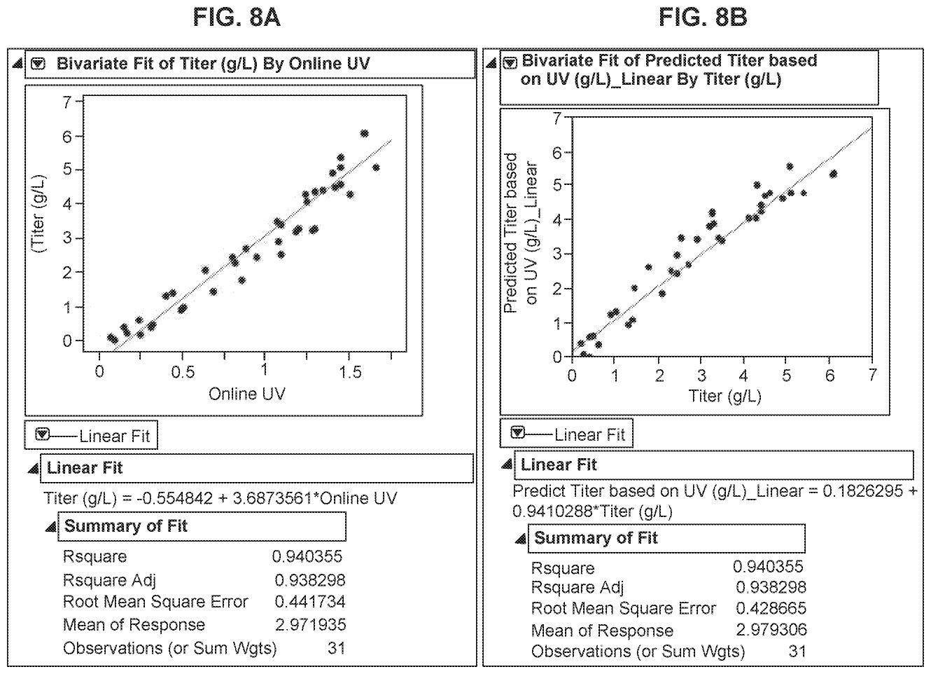

For incline portion of the data, a total of 22 samples were included in the model. Offline titer values were plotted against online UV values in FIG. 8. Linear fit was applied to the data. R² value for the linear fit was 0.98. Using this model, predicted titer values were calculated and compared with actual titer values. As shown in FIG. 8a and FIG. 8b, the slope of fit was very close to 1 and R² was 0.98.

To predict target protein concentration from start collection to end loading, a linear model was generated: Model Predicted Titer=a+b*(online UV signal). Model constants a and b are dependent on titer level. a=−0.35, b=2.88 if the titer is about 3.5 g/L or less; a=−0.69, b=4.06 if the titer is about 3.5 g/L or more.

B. Model fit for decline portion.

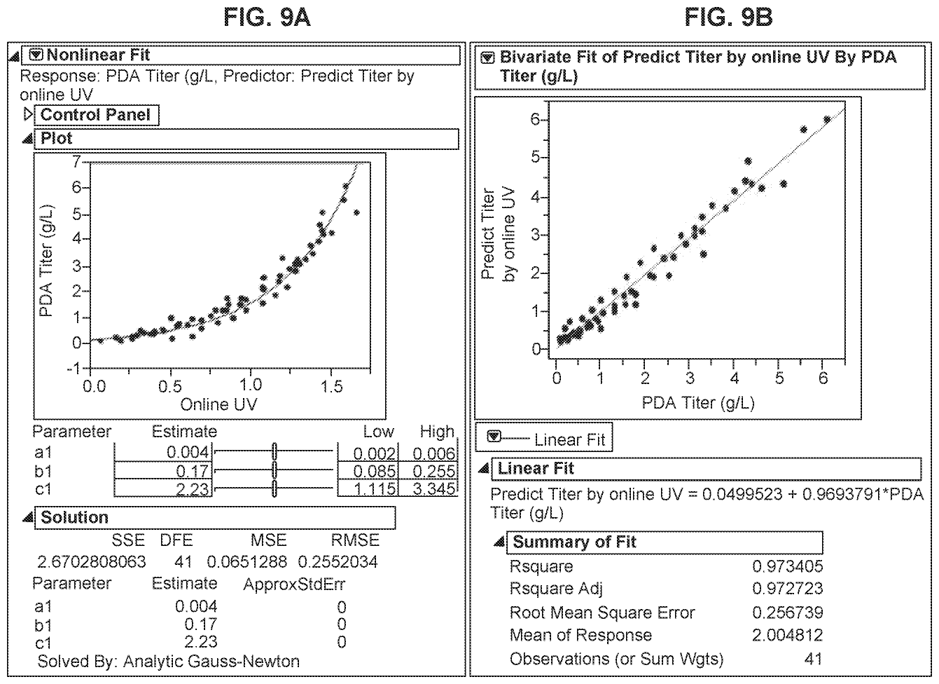

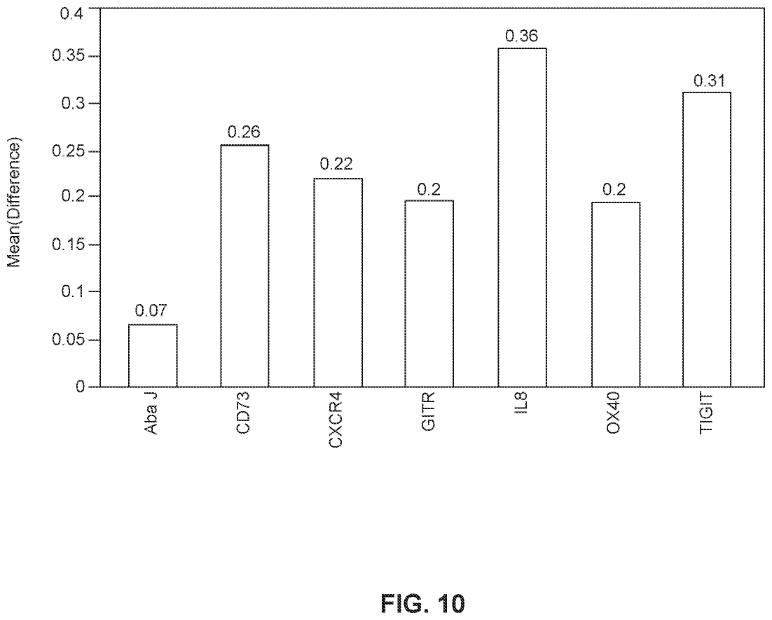

For decline portion of the data, a total of 41 samples were included in the model. Offline titer values were plotted against online UV values in FIG. 10. Non-linear fit was applied to the data. RMSE values for the non-linear fit were 0.26 and 0.04 for CHOZN and DG44 cell line respectively. Using this model, predicted titer values were calculated and compared with actual titer values (FIG. 9a and FIG. 9b). The slope of fit was close to 1 and R² was 0.97 and 0.99. FIGS. 9a and 9b.

To predict target protein concentration from start chasing to end collection, a non-linear model was generated: Model Predicted Titer=A+B*exp(C*online UV signal). Model constants A, B and C are dependent on titer level. A=−0.95, B=0.86, C=1.21 if the titer is about 3.5 g/L or less; A=0.02, B=0.13, C=2.41 if the titer is about 3.5 g/L or more.

### Example 5

### Test Model With Four at-Scale Cell Culture Processes

Four at scale (500L) cell culture processes (CD73, OX40, TIGIT and IL8) were harvested. The depth filters were scaled up based on small scale preliminary data. Offline samples after secondary depth filter were collected during the harvest processes for actual titer measurement. Online UV sensor values were entered into JMP software. Predicted titer values based on online UV sensor values, using the model, were calculated and compared with offline titer measurements.

TABLE 4
Cell culture process properties for molecules tested in this report

| | | Anti- | Anti- | Anti- | Anti- | Anti- | Anti- |
|---|---|---|---|---|---|---|---|
| | Aba J | GITR Ab | CXCR4 Ab | CD73 Ab | TIGIT Ab | OX40 Ab | IL8 Ab |
| Cell line | DG44 | CHOZN | CHOZN | CHOZN | CHOZN | DG44 | CHOZN |
| Product | Fusion Protein | IgG1 | IgG4 | IgG1/2 | IgG1 | IgG4 | IgG1 |
| | (CTLA-4 | | | hybrid | | | |
| | Ig molecule) | | | | | | |
| Peak viable cell | ~15 | ~30 | ~45 | ~20 | ~30 | ~15 | ~25 |
| density (×10\hat{ }6 | | | | | | | |
| cells/mL) | | | | | | | |
| Viability* on | 84% | 62% | 61% | 59% | 62% | 55% | 57% |
| harvest day | | | | | | | |
| Background | 80% | 76% | 68% | 77% | 73% | 80% | 76% |
| noise (HCP, | | | | | | | |
| media pigments) | | | | | | | |
| Titer (g/L) | 3-3.5 | 5.5-6 | 5-6 | 4.5-5 | 6-7 | 3 | 3.5-4.5 |
| Volume | 200 | 405 | 30 | 415 | 400 | 372 | 398 |
| harvested (L) | | | | | | | |
| Purpose | Model | Model | Model | Test | Test | Test | Test |

Note:
Viability here was calculated as follows: Viability (%) = VCD at harvest day/Peak VCD * 100%.

TABLE 5
Model fit evaluation for seven molecules studied

| | Aba | | | | | | |
|---|---|---|---|---|---|---|---|
| Molecule | NGP | GITR | CXCR4 | CD73 | TIGIT | OX40 | IL8 |
| Model Fit | 0.15 | 0.27 | 0.12 | 0.09 | 0.32 | 0.07 | 0.12 |
| RMSE (incline | | | | | | | |
| part) | | | | | | | |
| Model Fit | 0.04 | 0.12 | 0.25 | 0.14 | 0.12 | 0.05 | 0.13 |
| RMSE (decline | | | | | | | |
| part) | | | | | | | |

The models generated above were tested using four different at-scale (500 L) cell culture harvest processes with the start titer of 0-0.1 g/L and end titer of 0.1-0.2 g/L. The model can test as low as 0.01 g/L based on UV signal of 0.01 Au. Model predicted titer values were compared with actual titer values using JMP software. Model fit RMSE values for each process were shown in FIG. 10.

Difference between the model predicted and actual titer values were calculated. FIG. 12 shows the difference for each process tested. Overall mean difference ranges from 0.07-0.36 g/L, suggesting the models could be reliably applied to different processes with a variety of properties as shown in Table 2.

A. Using online sensors to control harvest process and improving harvest yield

TABLE 6
Yield improvement using new harvest skid for seven molecules studied

| | Aba | | | | | | |
|---|---|---|---|---|---|---|---|
| Molecule | NGP | GITR | CXCR4 | CD73 | TIGIT | OX40 | IL8 |
| Yield using old method | 90.0% | 86.3% | NA | 91.5% | 83.1% | 88.4% | 88.4% |
| Yield using new method | 93.7% | 91.6% | 93.6% | 93.7% | 85.3% | 91.6% | 93.7% |

Yield of harvest process was calculated using the following equation:

$$Yield = \frac{( \begin{matrix}Titer of clarified bulk ( g \text{/} L ) * \\ Volume of clarified bulk ( L )\end{matrix} ) * 100 \%}{\begin{matrix}Harvest day titer bioreactor ( g \text{/} L ) * \\ Harvest day bioreactor volume ( L )\end{matrix}}$$

As shown in Table 6, yield using the new harvest skid was 2-5% higher than that using the old method.

In this study, a real-time monitoring and controlling harvest skid was designed and examined for depth filtration harvest of several therapeutic proteins.

Online sensors were built into this harvest skid and their real-time readings were integrated into Delta V™ system to achieve automatic monitoring and control of critical process parameters. Model to translate online UV signal to real-time target protein concentration during different stages of harvest process was generated in a sequential of experiment steps: adjustment of UV sensor path-length, pure protein testing and complex cell culture sample testing. The model was then successfully tested with several at-scale harvest processes with a wide range of process properties, including background noise level, product level, total cell density and viability.

With this novel harvest skid and statistic model generated in this study, cell culture clarification process was monitored and controlled in a quantitative way, which significantly improved harvest robustness and protein yield. The online titer information itself is an important indicator of cell culture performance and can be used for immediate loading determination for Protein A chromatography in downstream processing.

### Example 6

### Real-Time Monitoring Process of a New Protein During Protein Harvest

A new target protein is selected to be clarified using this harvest skid. First, all sensors on the harvest skid are connected in-line as described in FIG. 2 to monitor pressure, flow, UV, turbidity and temperature during the harvest process. Second, water source is connected with LEVITRONIX® gravity pump. Total flow amount and flow rate are entered into Delta V™ to control the depth filter flushing step. After total flow amount is reached, bioreactor source is connected with LEVITRONIX® gravity pump to start loading of cell culture to depth filters. Third, UV prediction model constants for incline portion are entered into Delta V™; and start of collection cut-off threshold are entered into Delta V™. Online UV signal is translated to target protein concentration during loading. Once threshold is met, the receiving vessel is connected with the sterile filter to collect clarified bulk. Fourth, after bioreactor is emptied, PBS source is connected with LEVITRONIX® gravity pump to start chasing step. UV prediction model constants for decline portion are entered into Delta V™ based on cell line type; and end of collection cut-off threshold is entered into Delta V. Online UV signal is translated to target protein concentration during chasing. Once threshold is met, the receiving vessel is disconnected from the process stream. During the entire harvest process, pressure, turbidity and temperature is monitored to indicate out-of-control issue.

### Example 7

### Confirmation of Online Predicted Titer From Online UV Signal by Offline Titer Analysis

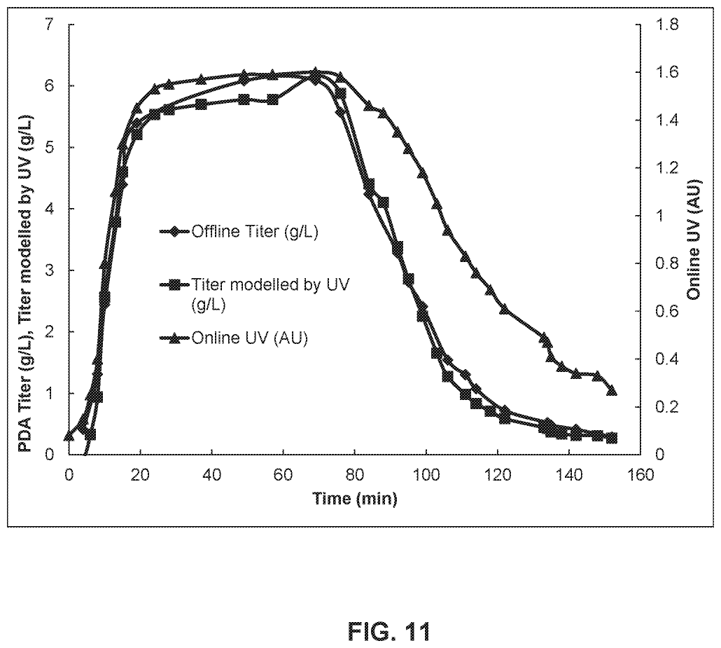

A harvest process started with a water-for-injection (WFI) flush of depth filters. A UV sensor was connected to the outlet of a secondary depth filter. Once the filtration became steady, a clear flow was seen from the outlet; the UV sensor was zeroed at that point. After the desired amount of WFI was flushed through the filter, cell culture media were connected with the filter inlet to begin the loading. The online UV trace of the media was monitored along the loading. The filtrate samples were taken along the loading and analyzed offline by a titer assay. FIG. 11 shows that the titer trace was achieved by modelling from the UV signal. The titer trace by modelling was well aligned with the offline titer assay results and therefore can be used to start and end the collection for process robustness and yield improvement.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, N.Y.); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

In some embodiments, disclosed herein is an apparatus for controlling, modulating, increasing, or improving protein yield in a sample mixture comprising a target protein and impurities. The apparatus may include one or more sensor. The sensors may comprise pressure sensors, UV sensors, turbidity sensors, temperature sensors, flow sensors, and any combination thereof.

In some embodiments, the apparatus is designed to integrate all sensors, including pressure (4), UV (1), turbidity (2), temperature (2) and flow sensor (1), into the apparatus. In some embodiments, the apparatus comprises three PMAT (Pressure Monitor Alarm Transmitter) controllers. In some embodiments, the PMAT controllers are built into the apparatus to accommodate a total of ten different sensors. In some embodiments, a gravity pump (e.g., a LEVITRONIX® gravity pump) is used to drive liquid to depth filters and is mounted in the apparatus. In some embodiments, the system is movable, lockable and/or e-stoppable. The apparatus may also comprise a processor configured to control collection of the target protein. The processor may also be configured to change a condition of the apparatus, for example, the temperature, pressure, turbidity, or flow. The processor may also be configured to control the collection of the target protein. In some embodiments, the processor may use an established model to determine a culture harvesting process. The cell culture harvesting process may comprise a filtration based cell culture harvesting process. The processor may be configured to use a target protein titer. The apparatus may be incorporate into a system for controlling, modulating, increasing, or improving protein yield in a sample mixture comprising a target protein and impurities.

All of the references cited above, as well as all references cited herein and the amino acid or nucleotide sequences (e.g., GenBank numbers and/or Uniprot numbers), are incorporated herein by reference in their entireties.

1. A method of monitoring in real-time a target protein concentration (titer) in a sample mixture comprising a target protein and impurities comprising monitoring in real-time an ultraviolet (UV) signal of the sample mixture and automatically transferring the UV signal into target protein titer using established models during a filtration based cell culture harvesting process.

2. A method of controlling target protein collection and improving protein yield in a sample mixture comprising a target protein and impurities comprising monitoring in real-time an ultraviolet (UV) signal of the sample mixture during a filtration based cell culture harvesting process.

3. The method of claim 1 or claim 2, wherein the UV signal is continuously transferred to a titer of the target protein according to established models and automatic control.

4. The method of claim 3, wherein the titer of the target protein is at least about 0.01 g/L, at least about 0.02 g/L, at least about 0.03 g/L, at least about 0.04 g/L, at least about 0.05 g/L, at least about 0.06 g/L, at least about 0.07 g/L, at least about 0.08 g/L, at least about 0.09 g/L, at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 6.5 g/L, at least about 7 g/L, at least about 7.5 g/L, at least about 8 g/L, at least about 8.5 g/L, at least about 9 g/L, at least about 9.5 g/L, at least about 10 g/L, at least about 10.5 g/L, at least about 11 g/L, at least about 11.5 g/L, at least about 12 g/L, at least about 12.5 g/L, at least about 13 g/L, at least about 13.5 g/L, at least about 14 g/L, at least about 14.5 g/L, at least about 15 g/L, at least about 15.5 g/L, at least about 16 g/L, at least about 16.5 g/L, at least about 17 g/L, at least about 17.5 g/L, at least about 18 g/L, at least about 18.5 g/L, at least about 19 g/L, at least about 19.5 g/L, or at least about 20 g/L.

5. The method of claim 3 or claim 4, further comprising beginning collection of the target protein when the titer is at least about 0.05 g/L, at least about 0.06 g/L, at least about 0.07 g/L, at least about 0.08 g/L, at least about 0.09 g/L, at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 6.5 g/L, at least about 7 g/L, at least about 7.5 g/L, at least about 8 g/L, at least about 8.5 g/L, at least about 9 g/L, at least about 9.5 g/L, at least about 10 g/L, at least about 10.5 g/L, at least about 11 g/L, at least about 11.5 g/L, at least about 12 g/L, at least about 12.5 g/L, at least about 13 g/L, at least about 13.5 g/L, at least about 14 g/L, at least about 14.5 g/L, at least about 15 g/L, at least about 15.5 g/L, at least about 16 g/L, at least about 16.5 g/L, at least about 17 g/L, at least about 17.5 g/L, at least about 18 g/L, at least about 18.5 g/L, at least about 19 g/L, at least about 19.5 g/L, or at least about 20 g/L.

6. The method of claim 5, wherein the titer that the target protein is collected is between about 0.05 g/L and about 20 g/L, between about 0.1 g/L and about 20 g/L, between about 0.2 g/L and about 20 g/L, between about 0.3 g/L and about 20 g/L, between about 0.4 g/L and about 20 g/L, between about 0.5 g/L and about 20 g/L, between about 0.6 g/L and about 20 g/L, between about 0.7 g/L and about 20 g/L, between about 0.8 g/L and about 20 g/L, between about 0.9 g/L and about 20 g/L, between about 1 g/L and about 20 g/L, between about 0.05 g/L and about 15 g/L, between about 0.1 g/L and about 15 g/L, between about 0.2 g/L and about 15 g/L, between about 0.3 g/L and about 15 g/L, between about 0.4 g/L and about 15 g/L, between about 0.5 g/L and about 15 g/L, between about 0.6 g/L and about 15 g/L, between about 0.7 g/L and about 15 g/L, between about 0.8 g/L and about 15 g/L, between about 0.9 g/L and about 15 g/L, or between about 1 g/L and about 15 g/L, between about 0.05 g/L and about 10 g/L, between about 0.1 g/L and about 10 g/L, between about 0.2 g/L and about 10 g/L, between about 0.3 g/L and about 10 g/L, between about 0.4 g/L and about 10 g/L, between about 0.5 g/L and about 10 g/L, between about 0.6 g/L and about 10 g/L, between about 0.7 g/L and about 10 g/L, between about 0.8 g/L and about 10 g/L, between about 0.9 g/L and about 10 g/L, or between about 1 g/L and about 10 g/L. The method of any one of claims 1 to 6, further comprising stopping the collection of the target protein when the collection titer is below about 0.1 or 0.2 g/L.

8. The method of any one of claims 1 to 7, wherein the target protein yield is increased at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20% compared to the protein yield without monitoring in real time an ultraviolet (UV) signal of the sample mixture.

9. The method of any one of claims 1 to 8, wherein the target protein is harvested from a culture medium having a cell density of at least about 1×10⁶ cells/mL, at least about 5×10⁶ cells/mL, at least about 1×10⁷ cells/mL, at least about 1.5×10⁷ cells/mL, at least about 2×10⁷ cells/mL, at least about 2.5×10⁷ cells/mL, at least about 3×10⁷ cells/mL, at least about 3.5×10⁷ cells/mL, at least about 4×10⁷ cells/mL, at least about 4.5×10⁷ cells/mL, or at least about 5×10⁷ cells/mL.

10. The method of any one of claims 1 to 9, wherein the protein filtration is a depth filtration.

11. The method of claim 10, wherein the depth filtration comprises a primary depth filter and/or a secondary depth filter.

12. The method of any one of claims 1 to 11, further comprising loading the sample mixture prior to the monitoring.

13. The method of any one of claims 1 to 12, further comprising flushing the depth filters with water or buffer before loading the cell culture and chasing the depth filters post loading the cell culture.

14. The method of any one of claims 1 to 13, further comprising chasing the sample mixture with phosphate buffered saline (PBS) or other buffers.

15. The method of any one of claims 1 to 14, wherein the filtration based cell culture harvesting process comprises a harvest skid.

16. The method of claim 15, wherein the harvest skid comprises a control system wherein the control system automatically starts collection of the protein when the set titer is achieved.

17. The method of claim 16, wherein the harvest skid comprises a control system, wherein the control system automatically stops collection of the protein when the set titer is achieved.

18. The method of claim 16 or 17, wherein the control system modulates flow rate of a liquid through the harvest skid.

19. The method of claim 18, wherein the control system automatically drives the pump to up-regulate flow rate through the harvest skid.

20. The method of claim 18, wherein the control system automatically drives the pump to down-regulate flow rate through the harvest skid.

21. The method of any one of claims 1 to 20, wherein the method does not comprise a step of air blow-down.

22. The method of any one of claims 1 to 21, wherein the target protein titer or the protein yield is not based on volume.

23. A method of increasing, controlling, or modulating protein yield in a sample mixture comprising a target protein and impurities comprising
a) flushing a harvest skid with water;
b) loading the sample into the harvest skid;
c) measuring an ultraviolet (UV) signal of the sample mixture during protein filtration in the harvest skid into a real-time protein titer;
d) starting collection of the protein based on the UV measurement and the real-time protein titer;
e) chasing the protein with PBS; and
f) stopping collection of the protein based on the UV measurement and the real-time protein titer;
wherein the UV signal correlates with the real-time protein titer during the filtration.

24. The method of any one of claims 1 to 23, further comprising measuring pressure, turbidity, temperature, flow rate, or any combination thereof.

25. The method of claim 24, further comprising measuring pressure using a pressure sensor.

26. The method of claim 25, wherein the pressure is measured in a range of −10 pounds per square inch (psi) to 50 psi, −10 psi to 40 psi, −9 psi to 40 psi, −8 psi to 40 psi, −7 psi to 30 psi, −6 psi to −20 psi, −7 psi to 40 psi, −8 psi to 40 psi, −9 psi to 45 psi, −10 psi to −45 psi, or −7 psi to −45 psi.

27. The method of claim 24, further comprising measuring turbidity.

28. The method of claim 27, wherein the turbidity is measured in a range of 0 absorbance units (AU) to 2 AU.

29. The method of claim 24, further comprising measuring temperature.

30. The method of claim 29, wherein the temperature is measured in a range of 0° C. to 70° C., 0° C. to 60° C., 0° C. to 50° C., 0° C. to 40° C., 5° C. to 70° C., 10° C. to 70° C., 15° C. to 70° C., 20° C. to 70° C., 10° C. to 60° C., 20° C. to 50° C., 20° C. to 40° C., 20° C. to 45° C., 30° C. to 40° C., 35° C. to 40° C., 20° C. to 30° C., 35° C. to 40° C., or 25° C. to 45° C,.

31. The method of claim 25, further comprising measuring flow.

32. The method of claim 31, wherein the flow is measured in a range of 0 L/min to 20 L/min, 0 L/min to 30 L/min, 0 L/min to 40 L/min, 0 L/min to 50 L/min, 0 L/min to 60 L/min, 0 L/min to 70 L/min, 0 L/min to 80 L/min, 0 L/min to 90 L/min, 0 L/min to 100 L/min, 0 L/min to 110 L/min, 0 L/min to 120 L/min, 0 L/min to 130 L/min, 0 L/min to 140 L/min, 0 L/min to 150 L/min, 0 L/min to 160 L/min, 0 L/min to 170 L/min, 0 L/min to 180 L/min, 0 L/min to 190 L/min, 0 L/min to 200 L/min, 0 L/min to 250 L/min, or 0 L/min to 300 L/min.

33. The method of any one of claims 1 to 32, wherein the harvest skid comprises one or more filters.

34. The method of claim 33, wherein the filters comprise a primary depth filter and a secondary depth filter.

35. The method of any one of claims 1 to 34, wherein the sample mixture is selected from the group consisting of a pure protein sample, a clarified bulk protein sample, a cell culture sample, and any combination thereof.

36. The method of any one of claims 1 to 35, wherein the protein is produced in culture comprising mammalian cells.

37. The method of claim 36, wherein the mammalian cells are Chinese hamster ovary (CHO) cells, HEK293 cells, mouse myeloma (NSO), baby hamster kidney cells (BHK), monkey kidney fibroblast cells (COS-7), Madin-Darby bovine kidney cells (MDBK) or any combination thereof.

38. The method of claim 37, wherein the mammalian cells are Chinese hamster ovary (CHO) cells.

39. The method of claim 38, wherein the CHO cells are selected from the group consisting of CHO-DG44 cells, CHOZN cells, CHO/dhfr-cells, CHOK1SV GS-KO cells, CHO-S cells.

40. The method of claim 38, wherein the mammalian cells are CHO-DG44, and wherein the target protein concentration is generated using a model predicted titer, wherein the model predicted titer comprises constants (a) and (b).

41. The method of any one of claims 38 to 40, wherein (a) is −0.35 and (b) is 2.88.

42. The method of claims 38 to 41, wherein the mammalian cells are CHO-DG44, and wherein the target protein concentration is generated using a model predicted titer, wherein the model predicted titer comprises constants (A), (B), and (C).

43. The method of claim 42, wherein (A) is −0.95, (B) is 0.86, and (C) is 1.21.

44. The method of claim 38, wherein the mammalian cells are CHOZN, and wherein the target protein concentration is generated using a model predicted titer, wherein the model predicted titer comprises constants (a) and (b).

45. The method of claim 44, wherein (a) is −0.69 and (b) is 4.06.

46. The method of any one of claims 38, 44, and 45, wherein the mammalian cells are CHOZN, and wherein the target protein concentration is generated using a model predicted titer, wherein the model predicted titer comprises constants (A), (B), and (C).

47. The method of claim 46, wherein (A) is 0.02, (B) is 0.13, and (C) is 2.41.

48. The method of any one of claims 1 to 47, wherein the protein comprises an antibody or a fusion protein.

49. The method of claim 48, wherein the protein is an anti-GITR antibody, an anti-CXCR4 antibody, an anti-CD73 antibody, an anti-TIGIT antibody, an anti-OX40 antibody, an anti-LAG3 antibody and anti-IL8 antibody.

50. The method of claim 48, wherein the protein is Abatacept or Belatacept.

51. A system for real time monitoring and controlling of protein yield, wherein the system comprises a sensor measuring a real-time UV signal of a sample mixture comprising a target protein and impurities.

52. The system of claim 51, wherein the system further comprises a sensor measuring pressure, turbidity, temperature, flow, weight, or any combination thereof.

53. The system of claim 51 or 52, for use in the method of any one of claims 1 to 50.

54. An apparatus comprising a sensor configured to measure a UV signal of a sample mixture comprising a target protein and impurities.

55. The apparatus of claim 54, further comprising a processor configured to control collection of the target protein.

56. The apparatus of any one of claims 54 and 55, wherein the processor is configured to use a target protein titer.

57. The apparatus of any one of claims 54 to 56, wherein the processor is configured to use established models to determine a cell culture harvesting process.

58. The apparatus of any one of claims 54 to 57, wherein the cell culture harvesting process comprises a filtration based cell culture harvesting process.

59. The system of any one of claims 51 to 53, wherein the system comprises the apparatus of any one of claims 54 to 58.
